(19) United States
(12) Patent Application Publication
(10) Pub. No.: US 2025/0152347 A1
(43) Pub. Date: May 15, 2025

(54) ATTACHMENT OF A MARKER TO A COMMISSURE OF A PROSTHETIC HEART VALVE

(71) Applicant: EDWARDS LIFESCIENCES CORPORATION, Irvine, CA (US)

(72) Inventors: Brendan Michael Dalbow, Oceanside, CA (US); Jeanette Jasmine Corona Kelly, Orange, CA (US)

(21) Appl. No.: 19/025,749

(22) Filed: Jan. 16, 2025

Related U.S. Application Data
(63) Continuation of application No. PCT/US2023/027977, filed on Jul. 18, 2023.
(60) Provisional application No. 63/368,859, filed on Jul. 19, 2022.

(51) Int. Cl. A61F 2/24

(52) U.S. Cl. CPC A61F 2/2418; A61F 2220/0075; A61F 2250/0098

(57) ABSTRACT

Methods and assemblies for attaching a radiopaque marker to an attachment member of a commissure of a prosthetic valve are disclosed. As one example, a prosthetic heart valve can comprise a frame including struts forming cells of the frame, at least one commissure comprising an attachment member and commissure tabs of two adjacent leaflets coupled thereto, the attachment member arranged across a cell of the frame and attached to struts defining the cell, and a radiopaque marker suspended within the cell and against the attachment member by sutures. The sutures extend from a first portion of the attachment member, through one or more apertures in the marker, and to a second portion of the attachment member, the sutures forming two knots in the second portion that are disposed on opposite sides of the marker such that the sutures form a non-crossing X-pattern through the marker and across the attachment member.

### CROSS-REFERENCE TO RELATED APPLICATIONS

This application is a continuation of PCT Application No. PCT/US2023/027977, filed Jul. 18, 2023, which claims the benefit of U.S. Provisional Patent Application No. 63/368,859, filed Jul. 19, 2022, the entire contents of each of which are incorporated herein by reference.

### FIELD

The present disclosure relates to methods and systems for attaching a marker to a commissure of a prosthetic heart valve.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

When deploying the prosthetic valve at the native valve, by inflating the balloon of the delivery device for example, the radially expanded prosthetic valve can be deployed at a random radial orientation relative to the native valve. It may be desirable to visualize a location of the commissures of the prosthetic valve during the implantation procedure in order to implant the prosthetic heart valve in a desired rotational alignment relative to the native valve. Thus, in some examples, the prosthetic valve can comprise a radiopaque marker secured to a commissure of the prosthetic heart valve in order to allow visualization of the commissure under imaging (for example, fluoroscopy) during the implantation procedure. In some instances, the marker can be sewn to an attachment member of a commissure of the prosthetic valve, where the attachment member is arranged across a cell of the frame of the prosthetic valve.

### SUMMARY

Described herein are prosthetic heart valves, delivery apparatuses, and methods for implanting prosthetic heart valves. Described also herein are methods for attaching a component, such as a radiopaque marker, to an attachment member of commissure of the prosthetic heart valve that is arranged across and secured to a cell of a frame of the prosthetic heart valve. In some examples, the methods can include using a plurality of sutures to suspend the marker within the cell, such that the marker remains taught against the attachment member without constraining the cell during radial compression and expansion of the frame. The disclosed prosthetic heart valves, delivery apparatuses, and methods can, for example, provide means of attaching radiopaque markers to a commissure attachment member or commissure arranged across a cell of a frame of the prosthetic heart valve that retain suture tension without limiting elongation of the frame as the frame is radially compressed (e.g., during crimping). As such, the devices and methods disclosed herein can, among other things, overcome one or more of the deficiencies of typical prosthetic heart valves and their delivery apparatus.

A commissure attachment member for a prosthetic device (such as a prosthetic valve) can comprise a main body portion and a radiopaque marker secured to the main body portion.

In some examples, the radiopaque marker is secured to the main body portion by sutures.

In some examples, the sutures extend from a first portion of the main body portion of the attachment member that is disposed adjacent to a first end of the marker, through one or more apertures in the marker, and to a second portion of the main body portion of the attachment member that is disposed adjacent to a second end of the marker.

In some examples, the sutures comprise two sutures.

In some examples, the sutures form a non-crossing X-pattern through the marker and across the main body portion of the commissure attachment member.

In some examples, a commissure attachment member comprises a main body portion, and a radiopaque marker secured to the main body portion by sutures that extend from a first portion of the main body portion of the attachment member that is disposed adjacent to a first end of the marker, through one or more apertures in the marker, and to a second portion of the main body portion of the attachment member that is disposed adjacent to a second end of the marker. The sutures form two connections to the second portion of the attachment member that are disposed on opposite sides of the marker such that the sutures form a non-crossing X-pattern through the marker and across the main body portion of the attachment member.

In some examples, a commissure attachment member comprises a main body portion, and a radiopaque marker secured to the main body portion by two sutures that extend through one or more apertures in the marker. The two sutures comprise a first suture that extends from a first location on the main body portion of the attachment member that is disposed axially offset from a first end of the marker and laterally offset from the marker in a first direction, through the one or more apertures in the marker, and to a second location on the main body portion of the attachment member that is disposed axially offset from a second end of the marker and laterally offset from the marker in the first direction, and a second suture that extends from a third location on the main body portion of the attachment member that is disposed axially offset from the first end of the marker and laterally offset from the marker in a second direction, through the one or more apertures in the marker, and to a fourth location on the main body portion of the attachment member that is disposed axially offset from the second end of the marker and laterally offset from the marker in the second direction.

In some examples, a commissure attachment member comprises one or more of the components recited in Examples 86-103 below.

A prosthetic heart valve can comprise a frame and a valvular structure coupled to the frame. In addition to these components, a prosthetic heart valve can further comprise one or more of the components disclosed herein.

In some examples, the prosthetic heart valve can comprise a sealing member configured to reduce paravalvular leakage.

In some examples, the frame is radially collapsible to a collapsed configuration and radially expandable to an expanded configuration.

In some examples, the frame includes a plurality of struts forming a plurality of cells of the frame arranged between an inflow end and an outflow end of the frame.

In some examples, the valvular structure includes a plurality of leaflets arranged within the frame and at least one commissure comprising an attachment member and commissure tabs of two adjacent leaflets coupled to the attachment member.

In some examples, the attachment member is arranged across a specified cell of the plurality of cells and is attached to struts of the frame defining the specified cell.

In some examples, the prosthetic heart valve can comprise a radiopaque marker suspended within the specified cell and disposed against the attachment member by sutures.

In some examples, the sutures extend from a first portion of the attachment member that is disposed adjacent to a first end of the marker, through one or more apertures in the marker, and to a second portion of the attachment member that is disposed adjacent to a second end of the marker.

In some examples, the sutures form two knots in the second portion of the attachment member that are disposed on opposite sides of the marker such that the sutures form a non-crossing X-pattern through the marker and across the attachment member.

In some examples, the sutures include a first suture that extends from a first location on the attachment member that is disposed axially offset from a first end of the marker and laterally offset from the marker in a first direction, through the one or more apertures in the marker, and to a second location on the attachment member that is disposed axially offset from a second end of the marker and laterally offset from the marker in the first direction, and a second suture that extends from a third location on the attachment member that is disposed axially offset from the first end of the marker and laterally offset from the marker in a second direction, through the one or more apertures in the marker, and to a fourth location on the attachment member that is disposed axially offset from the second end of the marker and laterally offset from the marker in the second direction.

In some examples, a prosthetic heart valve comprises a frame including a plurality of struts forming a plurality of cells of the frame arranged between an inflow end and an outflow end of the frame, where the frame is radially collapsible to a collapsed configuration and radially expandable to an expanded configuration. The prosthetic heart valve further comprises a plurality of leaflets arranged within the frame and at least one commissure comprising an attachment member and commissure tabs of two adjacent leaflets coupled to the attachment member. The attachment member is arranged across a specified cell of the plurality of cells and is attached to struts of the frame defining the specified cell. The prosthetic heart valve further comprises a radiopaque marker suspended within the specified cell and disposed against the attachment member by sutures that extend from a first portion of the attachment member that is disposed adjacent to a first end of the marker, through one or more apertures in the marker, and to a second portion of the attachment member that is disposed adjacent to a second end of the marker. The sutures form two knots in the second portion of the attachment member that are disposed on opposite sides of the marker such that the sutures form a non-crossing X-pattern through the marker and across the attachment member.

In some examples, a prosthetic heart valve comprises a frame including a plurality of struts forming a plurality of cells of the frame arranged between an inflow end and an outflow end of the frame, where the frame is radially collapsible to a collapsed configuration and radially expandable to an expanded configuration. The prosthetic heart valve further comprises a plurality of leaflets arranged within the frame, and at least one commissure comprising an attachment member and commissure tabs of two adjacent leaflets coupled to the attachment member. The attachment member is arranged across a specified cell of the plurality of cells of the frame and attached to struts of the frame forming the specified cell. The prosthetic heart valve further comprises a radiopaque marker suspended within the specified cell with two sutures that extend through one or more apertures in the marker. The two sutures comprise a first suture that extends from a first location on the attachment member that is disposed axially offset from a first end of the marker and laterally offset from the marker in a first direction, through the one or more apertures in the marker, and to a second location on the attachment member that is disposed axially offset from a second end of the marker and laterally offset from the marker in the first direction, and a second suture that extends from a third location on the attachment member that is disposed axially offset from the first end of the marker and laterally offset from the marker in a second direction, through the one or more apertures in the marker, and to a fourth location on the attachment member that is disposed axially offset from the second end of the marker and laterally offset from the marker in the second direction.

In some examples, a prosthetic heart valve comprises one or more of the components recited in Examples 120, 70-85, and 105 below.

An assembly can comprise a delivery apparatus and a prosthetic heart valve.

In some examples, the prosthetic heart valve is radially collapsible to a collapsed configuration and radially expandable to an expanded configuration.

In some examples, the collapsed prosthetic heart valve can be mounted around a valve mounting portion of a distal end portion of the delivery apparatus and radially expanded to the expanded configuration with the delivery apparatus inside a patient's body.

In some examples, the prosthetic heart valve includes one or more of the features of any one or more of the prosthetic heart valves described above.

In some examples, the prosthetic heart valve can comprise at least one commissure comprising an attachment member and commissure tabs of two adjacent leaflets coupled to the attachment member, where the attachment member is arranged across a specified cell of the frame and is attached to struts of the frame defining the cell.

In some examples, the prosthetic heart valve can comprise a radiopaque marker secured against the attachment member by first and second sutures that extend from opposite sides of a first portion of the attachment member that is disposed adjacent to a first end of the marker, through one or more apertures in the marker, and to a second portion of the attachment member that is disposed adjacent to a second end of the marker.

In some examples, tension of the first and second sutures against the marker is maintained when the frame of the prosthetic heart valve is in both the collapsed configuration and the expanded configuration.

In some examples, an assembly comprises a delivery apparatus and an implantable prosthetic heart valve that is radially collapsible to a collapsed configuration and radially expandable to an expanded configuration. The prosthetic heart valve comprises a frame including a plurality of struts forming a plurality of cells of the frame arranged between an inflow end and an outflow end of the frame, where the frame is radially collapsible to a collapsed configuration and radially expandable to an expanded configuration. The prosthetic heart valve further comprises a plurality of leaflets arranged within the frame and at least one commissure comprising an attachment member and commissure tabs of two adjacent leaflets coupled to the attachment member, where the attachment member is arranged across a specified cell of the plurality of cells of the frame and is attached to struts of the frame defining the specified cell. The prosthetic heart valve further comprises a radiopaque marker secured against the attachment member by first and second sutures that extend from opposite sides of a first portion of the attachment member that is disposed adjacent to a first end of the marker, through one or more apertures in the marker, and to a second portion of the attachment member that is disposed adjacent to a second end of the marker. The first and second sutures form first and second knots, respectively, in the second portion of the attachment member that are disposed on opposite sides of the marker such that tension of the first and second sutures against the marker is maintained when the frame is in both the collapsed configuration and the expanded configuration. The collapsed prosthetic heart valve can be mounted around a valve mounting portion of a distal end portion of the delivery apparatus and radially expanded to the expanded configuration with the delivery apparatus inside a patient's body.

In some examples, an assembly comprises one or more of the components recited in Examples 21-42 below.

In some examples, a method can comprise extending sutures from opposite sides of a first portion of a commissure attachment member, through one or more apertures in a marker disposed on the attachment member, and to opposite sides of a second portion of the attachment member.

In some examples, the first portion is disposed adjacent to a first end of the marker and the second portion is disposed adjacent to a second end of the marker, the second end arranged opposite the first end in an axial direction.

In some examples, the method can further comprise forming knots with the sutures on opposite sides of the second portion of the attachment member such that the marker is suspended within the cell.

In some examples, the method can further comprise radially compressing the prosthetic heart valve onto a delivery apparatus from a radially expanded configuration to a radially compressed configuration, and moving the knots axially away from the marker and laterally toward one another in response to axially elongating the frame during radial compression of the prosthetic heart valve such that a distance along the sutures between the knots and the marker is maintained between the radially expanded and radially compressed configurations.

In some examples, the method can further comprise securing commissure tabs of two adjacent leaflets of a prosthetic heart valve to a first surface of the attachment member to form a commissure, where the attachment member is configured to be arranged across a cell of a frame of the prosthetic heart valve.

In some examples, the radiopaque marker is positioned against a second surface of the attachment member that is disposed opposite the first surface.

In some examples, a method comprises extending sutures from opposite sides of a first portion of a commissure attachment member, through one or more apertures in a marker disposed on the attachment member, and to opposite sides of a second portion of the attachment member, where the first portion is disposed adjacent to a first end of the marker and the second portion is disposed adjacent to a second end of the marker, the second end arranged opposite the first end in an axial direction. The attachment member forms a commissure with two adjacent leaflets of a prosthetic heart valve and is arranged across a cell of a frame of the prosthetic heart valve. The method further comprises forming knots with the sutures on opposite sides of the second portion of the attachment member such that the marker is suspended within the cell, and radially compressing the prosthetic heart valve onto a delivery apparatus from a radially expanded configuration to a radially compressed configuration, and moving the knots axially away from the marker and laterally toward one another in response to axially elongating the frame during radial compression of the prosthetic heart valve such that a distance along the sutures between the knots and the marker is maintained between the radially expanded and radially compressed configurations.

In some examples, a method comprises securing commissure tabs of two adjacent leaflets of a prosthetic heart valve to a first surface of an attachment member to form a commissure, where the attachment member is configured to be arranged across a cell of a frame of the prosthetic heart valve. The method further comprises positioning a radiopaque marker against a second surface of the attachment member that is disposed opposite the first surface, and extending a first suture from a first location on the attachment member, through at least two apertures in the marker, and to a second location on the attachment member and forming a first knot with the first suture that secures the first suture to the first location of the attachment member. The first location is offset in a first axial direction and a first lateral direction from a first end of the marker and the second location is offset in a second axial direction and the first lateral direction from a second end of the marker. The method further comprises extending a second suture from a third location on the attachment member, through the at least two apertures in the marker, and to a fourth location on the attachment member and forming a second knot with the second suture that secures the second suture to the second location of the attachment member. The third location is offset in a first axial direction and a second lateral direction from the first end of the marker and the fourth location is offset in the second axial direction and the second lateral direction from the second end of the marker. The method further comprises securing the attachment member to struts of the frame that define the cell with a third suture such that the marker is disposed within the cell and not directly attached to the struts defining the cell.

In some examples, a method comprises one or more of the features recited in Examples 43-69 and 104 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1A is a perspective view of a prosthetic heart valve, according to one example.

FIG. 1B is a perspective view of the prosthetic valve of FIG. 1A with the components on the outside of the frame shown in transparent lines for purpose of illustration.

FIG. 2 is a side view of an example of a delivery apparatus configured to deliver and implant a radially expandable prosthetic heart valve at an implantation site.

FIG. 3 is a perspective view of a portion of an exemplary prosthetic valve with an attachment member of a commissure arranged across a cell of the prosthetic valve and secured to frame struts defining the cell, where the prosthetic valve is in a radially expanded configuration.

FIG. 4 is a perspective view of a portion of the prosthetic valve of FIG. 3 where the prosthetic valve is in a radially collapsed configuration.

FIG. 5 is an exploded view of a commissure for a prosthetic valve comprising an attachment member and commissure tabs of two adjacent leaflets.

FIG. 6 shows a side view of the attachment member of FIG. 5 secured to the commissure tabs of the leaflets, prior to finishing folding of the commissure tabs.

FIG. 7 is a top view of the assembled commissure of FIG. 5.

FIG. 8 is a bottom view of the assembled commissure of FIG. 5.

FIG. 9 shows an example of a marker positioned against an elongate or elongated flap of an exemplary attachment member, the attachment member configured to form a commissure with commissure tabs of adjacent leaflets of a prosthetic heart valve and configured to be arranged across a cell of a prosthetic heart valve and secured to struts forming the cell.

FIG. 10 is a schematic depicting a radiopaque marker secured to a commissure attachment member according to an exemplary sewing method with sutures forming a non-crossing X pattern through the marker, where the attachment member is in an axially shortened state.

FIG. 11 is a schematic depicting the radiopaque marker secured to the commissure attachment member of FIG. 10, where the attachment member is in an axially elongated state.

FIG. 12 is another schematic depicting the radiopaque marker secured to the commissure attachment member according to an exemplary sewing method with sutures forming a non-crossing X pattern through the marker, where the sutures are first used to secure commissure tabs of adjacent leaflets to the attachment member prior to securing the marker to the attachment member.

FIG. 13 is another view of the commissure of FIG. 12 illustrating the sutures extending through the marker and a flap of the attachment member in the non-crossing X pattern and a plurality of knots used to secure the marker and the flap to a main body portion of the attachment member.

FIG. 14A shows a front view of a cell of frame of a prosthetic valve in a radially expanded state, with the attachment member of FIGS. 12 and 13 secured to the struts defining the cell and the marker secured to the attachment member.

FIG. 14B shows a side view of the cell and attachment member of FIG. 14A.

FIG. 15 shows a front view of the prosthetic valve of FIG. 14A in a radially collapsed state.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

As used herein, “e.g.” means “for example,” and “i.e.” means “that is.”

### Overview of the Disclosed Technology

As introduced above, prosthetic heart valves can comprise a radiopaque marker secured to a commissure of the prosthetic heart valve to allow for visualization of the commissure under imaging (for example, fluoroscopy) during the implantation procedure. As a result, a user can determine a location of the commissure relative to the native anatomy (native heart valve) and more easily implant the prosthetic valve in a desired rotational, axial, and/or lateral orientation relative to the native heart valve. In some instances, the marker can be sewn to an attachment member of a commissure of the prosthetic valve, the attachment member arranged across a cell of the frame of the prosthetic valve.

The prosthetic valves disclosed herein can be radially compressible and expandable between a radially compressed (or collapsed) state and a radially expanded state. The prosthetic valves can be crimped on or retained by an implant delivery apparatus in the radially compressed state while being advanced through a patient's vasculature on the delivery apparatus. The prosthetic valve can be expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

When radially compressing or crimping the prosthetic valve around the delivery apparatus, into the radially compressed state, the frame of the prosthetic valve elongates in an axial direction, thereby laterally constricting the cells of the frame. In some examples, the radiopaque marker is sewn to the attachment member using sutures that are also used to secure the attachment member to frame struts defining the top and the bottom of the cell. In such instances, the marker essentially connects the top and bottom struts defining the cell together. Thus, when the prosthetic valve is crimped into the radially compressed state, the marker can prevent the frame from fully elongating (into the fully collapsed state). As a result, the prosthetic valve can have a larger crimp profile (larger diameter) on the delivery apparatus, thereby increasing push forces felt by a user as the prosthetic heart valve is advanced on the delivery apparatus to the implantation site.

Described herein are prosthetic heart valves, delivery apparatuses, and methods for assembling and implanting prosthetic heart valves. In some examples, such devices, apparatuses, and/or methods can provide a device and/or method for attaching a radiopaque marker to an attachment member of a commissure of a prosthetic heart valve that is arranged across a cell of a frame of the prosthetic heart valve, such that the marker can move freely as the frame elongates while still retaining tension of the marker against the attachment member. As a result, the frame can fully compress to its compressed or collapsed state and a crimp profile of the radially compressed prosthetic heart valve on the delivery apparatus can be minimized. As a result, push forces felt by a user advancing the prosthetic heart valve on the delivery apparatus to an implantation site can be reduced.

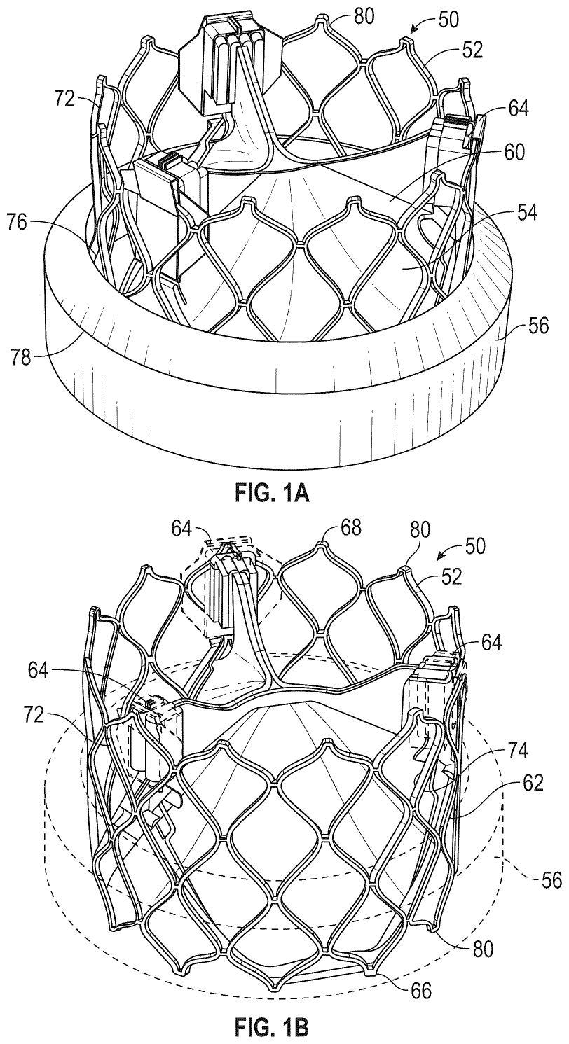

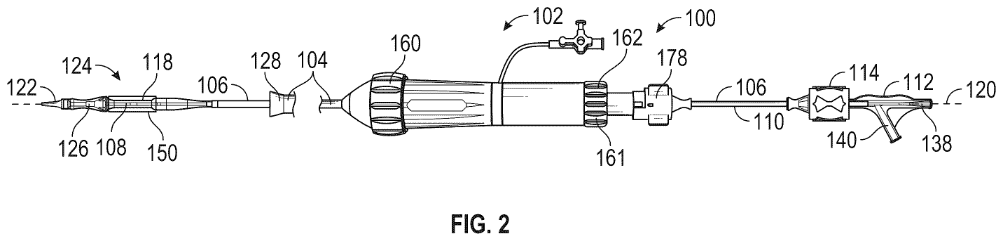

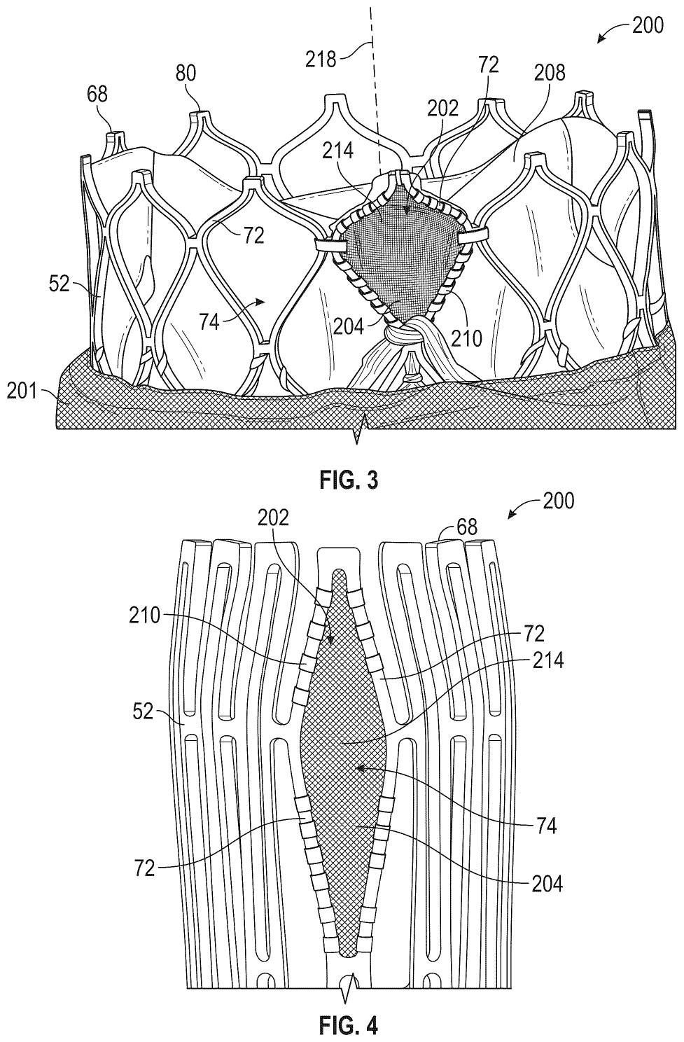

An exemplary prosthetic heart valve is shown in FIGS. 1A-1B. As introduced above, the prosthetic heart valve can be crimped on a delivery apparatus, such as the exemplary delivery apparatus shown in FIG. 2, in a radially compressed state while being advanced through a patient's vasculature on the delivery apparatus. The prosthetic heart valve can be radially expanded from the radially compressed state (FIG. 4) to a radially expanded state (FIG. 3) once the prosthetic heart valve reaches the implantation site. During radial compression of the prosthetic heart valve, it's frame radially compresses and elongates in an axial direction. As a result, cells defined by struts of the frame constrict and elongate.

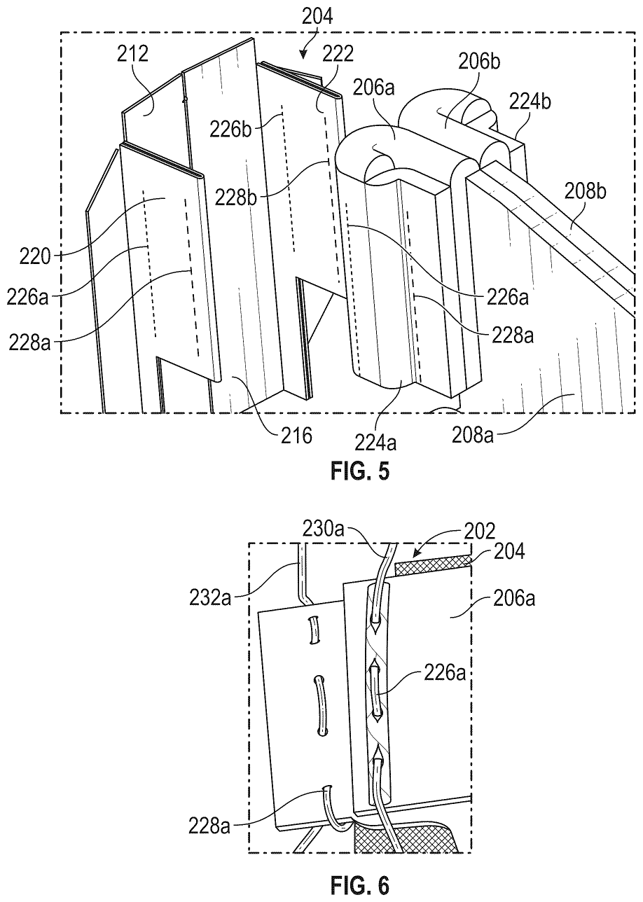

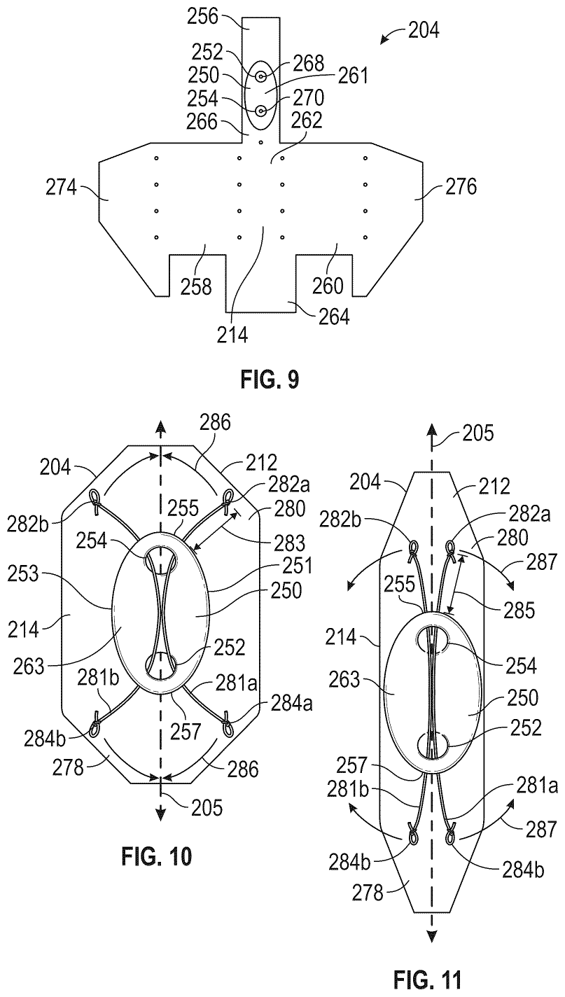

In some examples, as shown in FIGS. 5-8, commissures can be formed by attaching commissure tabs of adjacent leaflets together and to an attachment member (an exemplary attachment member is shown in FIG. 9). The commissures can then be attached to cells (for example, outflow cells) of the frame of the prosthetic heart valve by arranging each attachment member across a cell of the frame and securing the attachment member to struts defining the cell (as shown in FIGS. 3 and 4). In some instances, the sutures used to connect the commissure tabs and attachment member together can also be used to secure the attachment member to the struts of the cell.

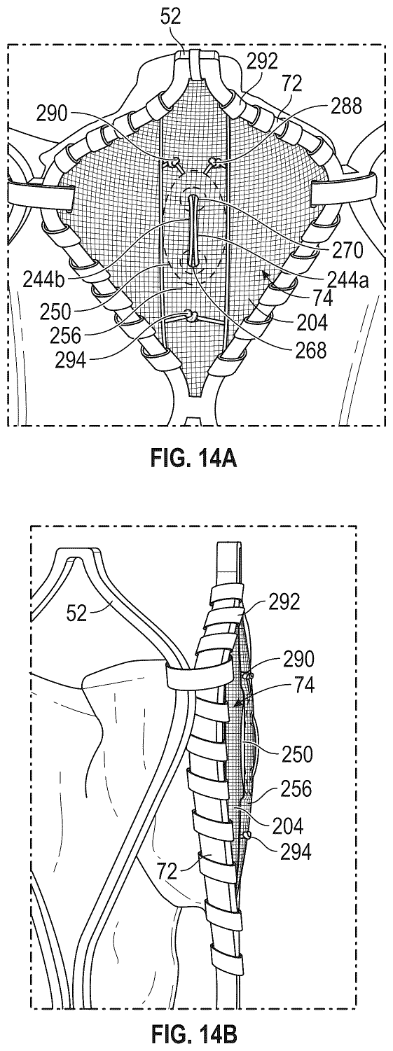

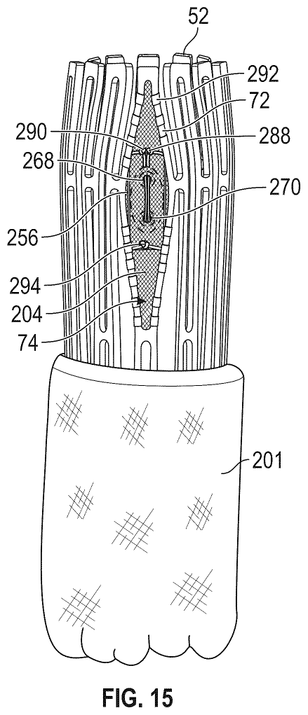

In order to avoid limiting elongation and compression of the frame during crimping of the frame onto the delivery apparatus, a radiopaque marker can be attached to the attachment member by sutures that suspend the marker across the cell, without directly suturing the marker to the struts forming the cell. For example, as shown in FIGS. 10-13, sutures from opposite sides of the attachment member can extend through one or more apertures in the marker and then can be knotted above the marker, on opposite sides of the marker and the attachment member. As a result, as the prosthetic valve is crimped, the knots can travel upwards and toward one another as the frame elongates, thereby retaining suture tension and holding the marker against the attachment member without limiting the elongation of the frame. Additional (different) sutures can then be used to secure the attachment member to the struts of the cell. The sutures suspending the marker in the cell form a non-crossing “X” shape (or a forward “C” and reverse “C” shape) through the marker and on the attachment member. Such a configuration allows the sutures to retain their tension, thereby maintaining the marker tight against the attachment member in both the radially expanded state (as shown in FIGS. 14A and 14B) and the radially compressed state (as shown in FIG. 15).

### Examples of the Disclosed Technology

FIGS. 1A and 1B show an exemplary prosthetic valve 50, according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

The prosthetic valve 50 can have three main components: a stent or frame, 52, a valvular structure 54, and a sealing member 56 (FIG. 1A). FIG. 1B is a perspective view of the prosthetic valve 50 with the components on the outside of the frame 52 (including the sealing member 56) shown in transparent lines for purposes of illustration. The prosthetic valve 50 can have an inflow end 66 and an outflow end 68.

The valvular structure 54 can comprise three leaflets 60, collectively forming a leaflet structure, which can be arranged to collapse in a tricuspid arrangement, although in other examples there can be greater or fewer number of leaflets (e.g., one or more leaflets 60). In some examples, the leaflets 60 can be formed of pericardial tissue (e.g., bovine pericardial tissue), biocompatible synthetic materials, or various other suitable natural or synthetic materials as known in the art and described in U.S. Pat. No. 6,730,118, which is incorporated by reference herein.

Each leaflet 60 can be coupled to the frame 52 along its inflow edge 62 (the lower edge in the figures; also referred to as “cusp edges”) and at commissures 64 of the valvular structure 54 where adjacent portions (e.g., commissure tabs) of two leaflets are connected to each other. In some examples, the commissures 64 can comprise an attachment member (e.g., comprising fabric, flexible polymer, or the like) arranged across a cell (e.g., commissure cell) of the frame 52, the cell formed by struts of the frame. The attachment member can be secured to the struts of the frame forming the cell and the adjacent portions of the two leaflets can be connected to the attachment member to form the commissure 64 (e.g., as shown in FIGS. 3-8, as described further below).

In some examples, a reinforcing element or connecting skirt, such as a fabric strip, can be connected directly to the cusp edges of the leaflets and to the struts of the frame to couple the cusp edges of the leaflets to the frame.

The frame 52 can be made of any of various suitable plastically-expandable materials (or example, stainless steel, etc.) or self-expanding materials (for example, Nitinol). When constructed of a plastically-expandable material, the frame 52 (and thus the prosthetic valve 50) can be crimped to a radially collapsed configuration on a delivery apparatus (e.g., catheter) and then expanded inside a patient by an inflatable balloon or equivalent expansion mechanism. Various crimping devices can be used to crimp the prosthetic valve 50 and the other prosthetic valves described herein around the delivery apparatus, such as the crimping devices described in U.S. Pat. No. 7,530,253, which is incorporated herein by reference.

In some instances, the prosthetic valve 50 can be crimped directly onto the inflatable balloon of the delivery apparatus, such that the prosthetic valve 50 is axially aligned with and disposed radially outward of the balloon during advancing the prosthetic valve on the delivery apparatus to the implantation site, for example as described in PCT Application No. PCT/US2021/047056, which is incorporated herein by reference. In alternate instances, the prosthetic valve 50 can be crimped onto the delivery apparatus axially offset from the balloon, and then moved over the balloon at the implantation site, prior to inflation of the balloon and radial expansion of the prosthetic valve, such as described in U.S. Pat. No. 9,339,384, which is incorporated herein by reference.

When constructed of a self-expandable material, the frame 52 (and thus the prosthetic valve 50) can be crimped to a radially collapsed configuration and restrained in the collapsed configuration by insertion into a sheath or equivalent mechanism of a delivery apparatus. Once inside the body, the prosthetic valve can be advanced from the delivery sheath, which allows the prosthetic valve to expand to its functional size.

Suitable plastically-expandable materials that can be used to form the frame 52 include, metal alloys, polymers, or combinations thereof. Example metal alloys can comprise one or more of the following: nickel, cobalt, chromium, molybdenum, titanium, or other biocompatible metal. In some examples, the frame 52 can comprise stainless steel. In some examples, the frame 52 can comprise cobalt-chromium. In some examples, the frame 52 can comprise nickel-cobalt-chromium. In some examples, the frame 52 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

The frame 52 in the illustrated example comprises a plurality of circumferentially extending rows of angled struts 72 defining rows of open cells 74 (or openings) of the frame. The frame 52 can have a cylindrical or substantially cylindrical shape having a constant diameter from the inflow end 66 to the outflow end 68 of the frame 52 as shown, or the frame 52 can vary in diameter along the height of the frame, as disclosed in U.S. Patent Publication No. 2012/0239142, which is incorporated herein by reference.

The frame 52, at each of the inflow end 66 and the outflow end 68, may comprise a plurality of apices 80 spaced apart from one another around a circumference of the frame 52.

The sealing member 56 in the illustrated example is mounted on the outside of the frame 52 and functions to create a seal against the surrounding tissue (e.g., the native leaflets and/or native annulus) to prevent or at least minimize paravalvular leakage. The sealing member 56 can comprise an inner layer 76 (which can be in contact with the outer surface of the frame 52) and an outer layer 78. The sealing member 56 can be connected to the frame 52 using suitable techniques or mechanisms. For example, the sealing member 56 can be sutured to the frame 52 via sutures that can extend around the struts 72 and through the inner layer 76. In alternative examples, the inner layer 76 can be mounted on the inner surface of the frame 52, while the outer layer 78 is on the outside of the frame 52.

The outer layer 78 can be configured or shaped to extend radially outward from the inner layer 76 and the frame 52 when the prosthetic valve 50 is deployed. When the prosthetic valve is fully expanded outside of a patient's body, the outer layer 78 can expand away from the inner layer 76 to create a space between the two layers. Thus, when implanted inside the body, this allows the outer layer 78 to expand into contact with the surrounding tissue.

Additional details regarding the prosthetic valve 50 and its various components are described in U.S. Patent Publication No. 2018/0028310, which is incorporated herein by reference.

FIG. 2 shows a delivery apparatus 100, according to an example, that can be used to implant an expandable prosthetic heart valve (e.g., the prosthetic valve 50 of FIGS. 1A and 1B and/or any of the other prosthetic heart valves described herein). In some examples, the delivery apparatus 100 is specifically adapted for use in introducing a prosthetic valve into a heart.

The delivery apparatus 100 in the illustrated example of FIG. 2 is a balloon catheter comprising a handle 102 and a steerable, outer shaft 104 extending distally from the handle 102. The delivery apparatus 100 can further comprise an intermediate shaft 106 (which also may be referred to as a balloon shaft) that extends proximally from the handle 102 and distally from the handle 102, the portion extending distally from the handle 102 also extending coaxially through the outer shaft 104. Additionally, the delivery apparatus 100 can further comprise an inner shaft 108 extending distally from the handle 102 coaxially through the intermediate shaft 106 and the outer shaft 104 and proximally from the handle 102 coaxially through the intermediate shaft 106.

The outer shaft 104 and the intermediate shaft 106 can be configured to translate (e.g., move) longitudinally, along a central longitudinal axis 120 of the delivery apparatus 100, relative to one another to facilitate delivery and positioning of a prosthetic valve at an implantation site in a patient's body.

The intermediate shaft 106 can include a proximal end portion 110 that extends proximally from a proximal end of the handle 102, to an adaptor 112. A rotatable knob 114 can be mounted on the proximal end portion 110 and can be configured to rotate the intermediate shaft 106 around the central longitudinal axis 120 and relative to the outer shaft 104.

The adaptor 112 can include a first port 138 configured to receive a guidewire therethrough and a second port 140 configured to receive fluid (e.g., inflation fluid) from a fluid source. The second port 140 can be fluidly coupled to an inner lumen of the intermediate shaft 106.

The intermediate shaft 106 can further include a distal end portion that extends distally beyond a distal end of the outer shaft 104 when a distal end of the outer shaft 104 is positioned away from an inflatable balloon 118 of the delivery apparatus 100. A distal end portion of the inner shaft 108 can extend distally beyond the distal end portion of the intermediate shaft 106.

The balloon 118 can be coupled to the distal end portion of the intermediate shaft 106.

In some examples, a distal end of the balloon 118 can be coupled to a distal end of the delivery apparatus 100, such as to a nose cone 122 (as shown in FIG. 2), or to an alternate component at the distal end of the delivery apparatus 100 (e.g., a distal shoulder). An intermediate portion of the balloon 118 can overlay a valve mounting portion 124 of a distal end portion of the delivery apparatus 100 and a distal end portion of the balloon 118 can overly a distal shoulder 126 of the delivery apparatus 100. The valve mounting portion 124 and the intermediate portion of the balloon 118 can be configured to receive a prosthetic heart valve in a radially compressed state. For example, as shown schematically in FIG. 2, a prosthetic heart valve 150 (which can be one of the prosthetic valves described herein) can be mounted around the balloon 118, at the valve mounting portion 124 of the delivery apparatus 100.

The balloon shoulder assembly, including the distal shoulder 126, is configured to maintain the prosthetic heart valve 150 (or other medical device) at a fixed position on the balloon 118 during delivery through the patient's vasculature.

The outer shaft 104 can include a distal tip portion 128 mounted on its distal end. The outer shaft 104 and the intermediate shaft 106 can be translated axially relative to one another to position the distal tip portion 128 adjacent to a proximal end of the valve mounting portion 124, when the prosthetic valve 150 is mounted in the radially compressed state on the valve mounting portion 124 (as shown in FIG. 2) and during delivery of the prosthetic valve to the target implantation site. As such, the distal tip portion 128 can be configured to resist movement of the prosthetic valve 150 relative to the balloon 118 proximally, in the axial direction, relative to the balloon 118, when the distal tip portion 128 is arranged adjacent to a proximal side of the valve mounting portion 124.

An annular space can be defined between an outer surface of the inner shaft 108 and an inner surface of the intermediate shaft 106 and can be configured to receive fluid from a fluid source via the second port 140 of the adaptor 112. The annular space can be fluidly coupled to a fluid passageway formed between the outer surface of the distal end portion of the inner shaft 108 and an inner surface of the balloon 118. As such, fluid from the fluid source can flow to the fluid passageway from the annular space to inflate the balloon 118 and radially expand and deploy the prosthetic valve 150.

An inner lumen of the inner shaft can be configured to receive a guidewire therethrough, for navigating the distal end portion of the delivery apparatus 100 to the target implantation site.

The handle 102 can include a steering mechanism configured to adjust the curvature of the distal end portion of the delivery apparatus 100. In the illustrated example, for example, the handle 102 includes an adjustment member, such as the illustrated rotatable knob 160, which in turn is operatively coupled to the proximal end portion of a pull wire. The pull wire can extend distally from the handle 102 through the outer shaft 104 and has a distal end portion affixed to the outer shaft 104 at or near the distal end of the outer shaft 104. Rotating the knob 160 can increase or decrease the tension in the pull wire, thereby adjusting the curvature of the distal end portion of the delivery apparatus 100. Further details on steering or flex mechanisms for the delivery apparatus can be found in U.S. Pat. No. 9,339,384, which is incorporated by reference herein.

The handle 102 can further include an adjustment mechanism 161 including an adjustment member, such as the illustrated rotatable knob 162, and an associated locking mechanism including another adjustment member, configured as a rotatable knob 178. The adjustment mechanism 161 is configured to adjust the axial position of the intermediate shaft 106 relative to the outer shaft 104 (e.g., for fine positioning at the implantation site). Further details on the delivery apparatus 100 can be found in PCT Application No. PCT/US2021/047056, which is incorporated by reference herein.

FIGS. 3 and 4 show partial views of another exemplary prosthetic valve 200 comprising the frame 52 in a radially expanded configuration (FIG. 3) and a radially compressed (or collapsed) configuration (FIG. 4). The prosthetic valve 200 can be similar to the prosthetic valve 50 of FIGS. 1A and 1B, except it includes an exemplary outer skirt 201 disposed around an outer surface of the frame 52 and comprises commissures 202 formed by securing connecting portions of adjacent leaflets 208 (commissure tabs) to a radially inward-facing surface of an attachment member 204 which is arranged across a cell 74 of the frame 52 and secured to struts 72 defining the cell 74 (for example, with sutures 210). The attachment member 204 can comprise a fabric, a flexible polymeric material, or a combination thereof. In some examples, the commissures 202 can be used in lieu of the commissures 64 in the prosthetic valve 50.

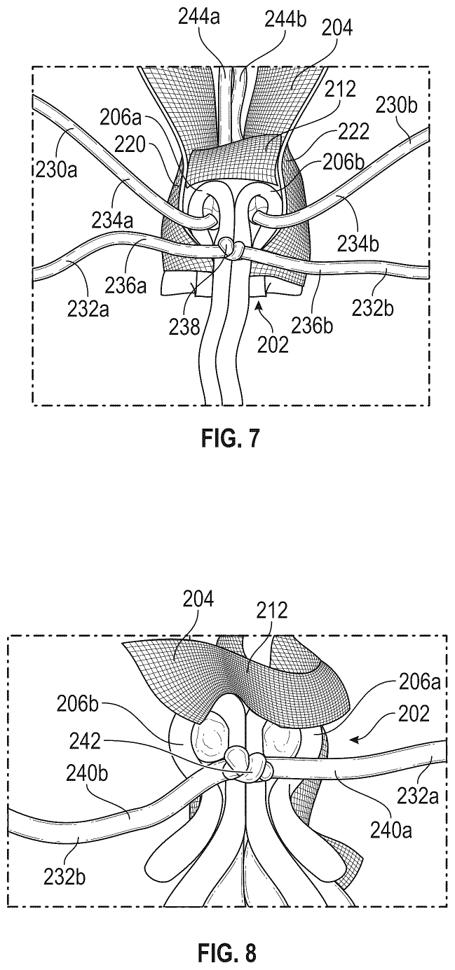

Detailed views depicting assembly of a commissure 202 is shown at FIGS. 5-8. FIG. 5 shows an exploded view of the commissure 202, prior to securing the attachment member 204 to the commissure tabs of the adjacent leaflets 208. FIG. 6 shows a side view of the attachment member 204 secured to the commissure tabs of the leaflets 208, prior to finishing folding of the commissure tabs, and FIGS. 7 and 8 show top and bottom views, respectively, of the assembled commissure 202. As used herein with reference to the commissures 202, the “top” or “top view” can refer to an end of the commissure disposed closest to the outflow end 68 of the frame 52 (compared to the opposite, bottom end) and the “bottom” or “bottom view” can refer to an end of the commissure disposed closer to the inflow end 66 of the frame 52 than the top end.

Additionally, an example of the attachment member 204 in a flat and unfolded state (prior to folding and commissure formation) is shown in FIG. 9. It should be noted that, in alternate examples, the attachment member 204 can have different configurations than shown in FIG. 9, such as not including the flap 256 or having a different overall shape (for example, a rectangular shape, a hexagonal shape, or the like).

To form each commissure 202, first and second commissure tabs 206a, 206b of adjacent first and second leaflets 208a, 208b can be folded and disposed adjacent to one another (FIG. 5). In some instances, the attachment member 204 can also be folded into a selected shape and comprise one or more folds. For example, as shown in FIGS. 3-5, the attachment member 204 can comprise a main body portion 212 which has a radially outward facing surface 214 (FIGS. 3 and 4) and a radially inward facing surface 216 that is configured to receive the folded commissures tabs 206a, 206b thereon (FIG. 5). The main body portion 212 can be shaped to fit within the cell 74 of the frame 52. The attachment member 204 can also comprise a first folded side portion 220 and a second folded side portion 222 arranged opposite one another across the main body portion 212 and which extend radially inward from the radially inward facing surface 216 toward a central longitudinal axis 218 of the prosthetic valve 200. In some examples, each of the first folded side portion 220 and the second folded side portion 222 can comprise two layers of the attachment member 204 (due to the fold).

After forming the folds, the folded commissure tabs 206a, 206b can then be positioned against the radially inward facing surface 216 with the first folded side portion 220 extending over an outer fold 224a of the first commissure tab 206a and the second folded side portion 222 extending over an outer fold 224b of the second commissure tab 206b. The folded commissure tabs 206a, 206b can be secured to the attachment member 204 by one or more stitch lines comprising a plurality of stitches of a suture (e.g., in-and-out stitches). FIG. 5 depicts two sets of dashed lines on the attachment member 204 and the folded commissure tabs 206a, 206b which represent two stitch lines on either side of the commissure 202 that are used to secure the attachment member 204 and the commissure tabs 206a, 206b together (dashed lines are shown on both the attachment member 204 and the commissure tabs 206a, 206b in the exploded view of FIG. 5, but a single stitch line can be formed at the same location on the attachment member 204 and commissure tabs when the commissure tabs 206a, 206b are arranged against the attachment member 204, as shown in FIG. 6).

More specifically, the stitch lines can include a primary stitch line 226a that extends through the first folded side portion 220 and the folded commissure tab 206a adjacent to the main body portion 212 (formed with a primary suture) and a primary stitch line 226b that extends through the second folded side portion 222 and the folded commissure tab 206b adjacent to the main body portion 212 (formed with another primary suture). The stitch lines can further include a secondary stitch line 228a that extends through the first folded side portion 220 and the folded commissure tab 206a radially inward of the main body portion 212 and the primary stitch line 226a (formed with a secondary suture) and a secondary stitch line 228b that extends through the second folded side portion 222 and the folded commissure tab 206b radially inward of the main body portion 212 and the primary stitch line 226b (formed with another secondary suture).

As shown in FIG. 6, the primary stitch line 226a can be formed with a primary suture 230a and the secondary stitch line 228a can be formed with a secondary suture 232a. Likewise, the primary stitch line 226b can be formed with a primary suture 230b and the secondary stitch line 228b can be formed with a secondary suture 232b. In some examples, the outer folds 224a and 224b can be formed by folding the commissure tabs 206a, 206b around the primary stitch lines 226a, 226b, such that the primary stitch lines 226a, 226b are tucked inside the commissure 202 (as shown in the top view of FIG. 7). Additional examples of commissure attachment members and methods for folding the leaflet commissure tabs and attachment member to form a commissure can be found in PCT Application No. PCT/US2022/012873, which is incorporated herein by reference.

The formation of the primary stitch lines 226a, 226b and the secondary stitch lines 228a, 228b with the primary sutures 230a, 230b and the secondary sutures 232a, 232b, respectively, can result in primary suture tails and secondary sutures tails formed at both the first or top end (FIG. 7) and the second or bottom end (FIG. 8) of the assembled commissure 202 (the primary suture tails are not visible in the view of FIG. 8). For example, as shown in FIG. 7, top primary suture tails 234a, 234b can extend out from a middle of the commissure 202, on opposite sides of the commissure 202 (and adjacent to the main body portion 212 of the attachment member 204) and top secondary suture tails 236a, 236b can be knotted together at the top ends of the commissure tabs 206a, 206b and extend away from the formed knot 238. As shown in FIG. 8, bottom secondary suture tails 240a, 240b can be knotted together at bottom ends of the commissure tabs 206a, 206b and extend away from the formed knot 242. In some examples, the bottom primary suture tails 244a, 244b can extend through the main body portion 212 of the attachment member 204, outward from the radially outward facing surface 214 (for example, as seen in FIG. 7).

The primary suture tails 234a, 234b, 244a, 244b, and the secondary suture tails 236a, 236b, 240a, 240b can be used secure additional components to the attachment member 204 and/or secure the attachment member 204 to the frame 52. Such additional components can comprise a radiopaque marker that is configured to be visualized under medical imaging, such as fluoroscopy.

For example, it may be desirable to visualize a location of one or more commissures of the prosthetic valve during an implantation procedure at a native valve in order to implant the prosthetic valve in a desired position relative to the native valve (such as a desired rotational alignment and/or axial position). In some examples, it may be desirable to implant the prosthetic valve such that commissures of the prosthetic valve align with commissures of the native valve. By attaching a radiopaque marker to one or more of the commissures of the prosthetic valve, a location of the commissures relative to the native valve (or alternate native anatomy) can be easily visualized under medical imaging (for example, fluoroscopy) during the implantation procedure, thereby facilitating rotational positioning of the prosthetic valve relative to the native valve.

An exemplary radiopaque marker 250 (referred to herein as a marker) that can be secured to the attachment member 204 is shown in FIGS. 9-11. The marker 250 can be configured to be visible under medical imaging. For example, the marker 250 can comprise a radiopaque material that is configured to be visible under medical imaging, such as fluoroscopy and/or other types of X-ray imaging. In some examples, the marker 250 can comprise a radiopaque or other material that is configured to be visible under MRI, ultrasound, and/or echocardiogram. In some examples, the marker 250 can comprise tantalum. In some examples, the marker 250 can comprise another type of radiopaque material or combination of materials, such as one or more of iodine, barium, barium sulfate, tantalum, bismuth, or gold.

The marker 250 is depicted in FIG. 9 (and FIGS. 10-13) as having an oblong or oval shape. However, in alternate examples, the marker can have a different shape, such as rectangular, arc-shaped, ring-shaped, circular, or having a shape of a letter of the alphabet (such as a “C”, “E”, “e”, or the like).

As shown in FIG. 9, the marker 250 has two apertures 252, 254 that are spaced axially apart from one another. However, in alternate examples, the marker 250 can have one or more apertures, such as two, three, four apertures, or the like. In still other examples, the apertures 252, 254 can be larger or smaller and/or have a different shape than shown in FIG. 9 (e.g., square, oblong, or hexagonal).

In order to attach the marker 250 to the attachment member 204, the marker 250 can first be positioned against an elongate flap 256 of the attachment member 204 (FIG. 9). For example, as shown in FIG. 9, the attachment member 204 can comprise a first side portion 258 and a second side portion 260 projecting laterally from a central portion 262 (or central region). In some examples, the attachment member 204 can further comprise a tab 264 projecting from an opposite edge of the central portion 262 than the flap 256.

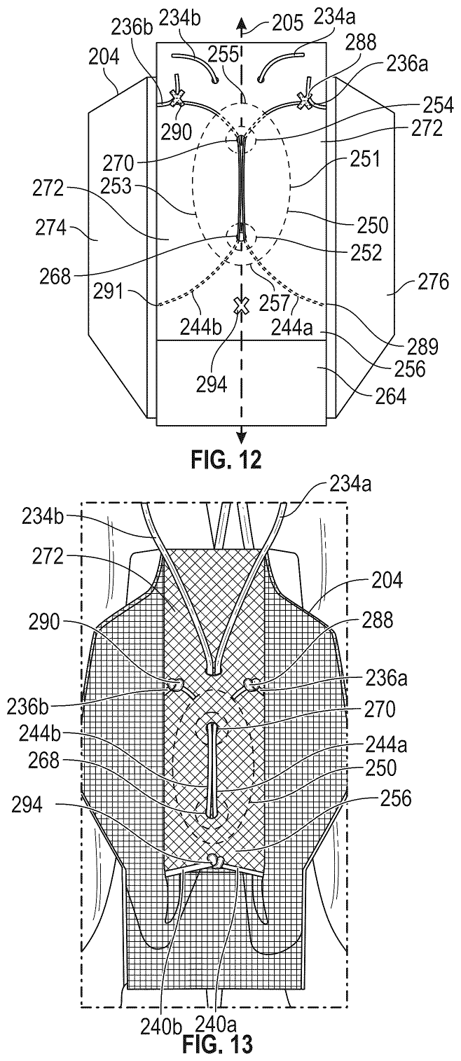

The marker 250 can be placed on a first surface 266 of the flap 256, over apertures 268, 270 in the flap 256 such that its apertures 252, 254 overlap the apertures 268, 270. In alternate examples, the flap 256 may not include pre-formed apertures 252, 254. The flap 256 can then be folded over the radially outward facing surface 214 of the central portion 262 of the attachment member 204. As such, the marker 250 is sandwiched between a second (outer) surface 272 of the flap 256 (shown in FIG. 13) and the radially outward facing surface 214 of the central portion 262 of the attachment member 204 (FIGS. 12 and 13).

In alternate examples, the attachment member may not include the flap 256. In such examples, the marker 250 can be placed against the radially outward facing surface 214 of the central portion 262 of the attachment member, without a flap covering the marker 250. The same or similar method can then be implemented, as described further below with reference to FIGS. 10-13.

As introduced above with reference to FIGS. 5-8, the attachment member 204 can be folded such that the main body portion 212, the first folded side portion 220, and the second folded side portion 222 shown in FIG. 5 are formed. By folding the first side portion 258 and the second side portion 260 of the attachment member 204, the first folded side portion 220 and the second folded side portion 222 can be formed (as shown in FIG. 5, for example) and the main body portion can then be defined by the central portion 262 and first and second edge portions 274, 276 of the respective first and second side portions 258, 260.

The marker 250 can be attached to the attachment member 204 using a sewing method that extends sutures through the marker 250 and forms knots on both sides of the attachment member 204, at locations that are spaced away from the marker 250, thereby suspending the marker 250 in the cell of the frame of the prosthetic valve and maintaining suture tension in both the radially expanded and compressed configurations of the frame, when the attachment member 204 is secured to the struts defining the cell. FIGS. 10 and 11 are schematics presenting a high level representation of a method for suturing a marker (or alternate component) to an attachment member. Though FIGS. 10 and 11 depict sewing the marker 250 to the attachment member 204, the method described below with reference to FIGS. 10 and 11 (and FIGS. 12 and 13) can be applied to alternate attachment members (e.g., attachment members having alternate shapes, and/or including or not including a flap) and/or alternate markers or components configured to be secured to the attachment member. In FIGS. 10 and 11, the flap 256 is omitted for ease of illustration. However, the flap 256 is shown in the more detailed views of FIGS. 12 and 13, as described further below. FIG. 12 is a schematic presenting a more specific example of the method introduced in FIGS. 10 and 11, where the primary and secondary sutures tails used to form the commissure 202 are used to suture the marker 250 to the attachment member 204.

Turning first to FIGS. 10 and 11, the marker 250 is shown disposed against the radially outward facing surface 214 of the attachment member 204. When the attachment member 204 is coupled to a cell of the prosthetic valve frame (e.g., an outflow cell), the outflow end 255 of the marker 250 can be directed toward the outflow end of the frame and the inflow end 257 of the marker 250 can be directed toward the inflow end of the frame (e.g., as shown in FIGS. 14A-15). As shown in FIGS. 10 and 11, two sutures 281a, 281b (which in some examples can be the bottom primary suture tails 244a, 244b, as described further below with reference to FIGS. 12 and 13) can extend from knots 284a, 282b (or other types of connection points or connections) in an inflow portion 278 (or first portion) of the attachment member 204, underneath the marker 250 (e.g., over a first radial surface of the marker 250), through a first aperture 252 of the marker 250, over the marker 250 between the first aperture 252 and a second aperture 254 (e.g., over a second radial surface 263 of the marker 250), through the second aperture 252, underneath the marker 250, and to an outflow portion 280 (second portion) of the attachment member 204 where two knots 282a, 282b are formed on opposite sides of the attachment member 204 (across the marker 250). In alternate examples, the knots 284a, 284b or the knots 282a, 282b can be replaced by points in the attachment member 204 through which the sutures 281a, 281b extend from behind the attachment member 204 to the radially outward facing surface 214 (without tying or knotting the suture).

The ends of the suture 281a (shown by knots 284a and 282a) can be disposed on a first lateral side of the attachment member 204, laterally offset from a first lateral side 251 of the marker 250, thereby substantially forming a forward “C” shape. Conversely, the ends of the suture 281b (shown by knots 284b and 282b) can be disposed on a second lateral side of the attachment member 204, laterally offset from a second lateral side 253 of the marker 250, thereby substantially forming a reverse “C” shape. As a result, the two sutures 281a, 281b form a non-crossing X-pattern through the marker 250 and across the attachment member 204.

It should be noted that, as used herein, a “non-crossing X-pattern” can refer to the forward C and reverse C pattern shown in FIGS. 10-13 where ends of the same suture or suture tail are disposed on the same (rather than opposite) sides of the marker and attachment member 204 (relative to a central longitudinal axis 205 of the attachment member 204). In some examples, an intermediate portion of the sutures or suture tails, which extend between the first and second apertures 252 and 254, for example, can cross or overlap one another, while still maintaining the overall non-crossing X-pattern of the sutures or suture tails.

As described further below, the non-crossing X-pattern described above can advantageously maintain outward suture tension in the sutures 281a, 281b as the attachment member 204 stretches and elongates axially (for examples, as the frame radially compresses). However, in alternative examples, the two sutures 281a, 281b may form a crossing X-pattern through the maker 250. For example, opposing first and second ends of the suture 281a can be disposed on opposite sides of the marker 250 and opposing first and second ends of the suture 281b can be disposed on opposite sides of the marker 250. In some instances, this crossing X-pattern may not maintain tension in the sutures 281a, 281b as the frame radially expands and compresses as well as the non-crossing X pattern described further below.

The non-crossing X sewing pattern shown in FIGS. 10 and 11 allows suture tension to be maintained when the prosthetic valve frame and attachment member 204 are in the radially expanded state (FIG. 10) and when the frame and attachment member 204 are in the radially compressed state (FIG. 11). For example, the knots 282a, 282b and 284a, 284b (or ends) of the sutures 281a, 281b move laterally towards one another and axially away from the outflow end 255 and inflow end 257 of the marker 250, respectively, as shown by arrows 286, as the frame is radially compressed (e.g., crimped) and the attachment member 204 elongates axially (FIG. 10). Similarly, as the frame is radially expanded, the knots 282a, 282b and 284a, 284b (or ends) of the sutures 281a, 281b move laterally away from one another and axially toward the outflow end 255 and inflow end 257 of the marker 250, respectively, as shown by arrows 287, as the attachment member 204 shortens axially (FIG. 11).

In this way, the knots 282a, 282b and 284a, 284b (or ends) of the sutures 281a, 281b travel with the frame and attachment member 204 as they axially elongate (during crimping) and axially shorten (during expansion). This allows a first distance 283 (or suture length) along the suture 281a or 281b, between each knot 282a, 282b and 284a, 284b and the end 255 or 257 of the marker 250, in the expanded state (FIG. 10) to be the same as (or substantially equal to) a second distance 285 (or suture length) along the suture 281a or 281b, between the same knot 282a, 282b and 284a, 284b and end 255 or 257 of the marker 250, in the compressed state (FIG. 11). By maintaining the first distance 283 and second distance 285, the sutures 281, 281b can retain a specified tension on the marker 250 through the crimping and expansion process, thereby keeping the marker 250 positioned against the attachment member 204 (e.g., without it bulging outward as the frame compresses or expands). Additionally, since the sutures 281a, 281b do not attach directly to the struts defining the cell across which the attachment member 204 is positioned (as described further below with reference to FIGS. 12-15), the frame can elongate as needed during radial compression of the frame.

FIG. 12 is a schematic depicting a more specific example of the sewing method introduced above with reference to FIGS. 10 and 11, which uses the primary and secondary sutures tails used to form the commissure 202 (as shown in FIGS. 5-8) to attach the marker 250 to the attachment member 204 and suspend the marker 250 across the cell of the frame of the prosthetic valve. FIG. 13 shows a view of the attachment member 204 after following the sewing method described below with reference to FIG. 12 (e.g., shows the formed knots, as described below). Additionally, FIGS. 14A, 14B, and 15 show different views of a cell 74 of the frame 52 with the attachment member 204 secured to the struts 72 defining the cell 74 and the marker 250 secured to the attachment member 204 (and not directly to the struts 72), according the method described below with reference to FIG. 12.

As introduced above with reference to FIG. 9, the marker 250 can be arranged against a first surface 266 of the flap 256 of the attachment member 204. The flap can then be folded over the central portion 262 of the attachment member 204 such that an opposite, second surface 272 of the flap 256 becomes an outer or radially outward facing surface of the attachment member 204 (when arranged in the cell of the frame of the prosthetic valve) and the marker 250 is covered by the flap 256. Thus, the marker 250 is shown with dashed lines in FIG. 12 (and FIGS. 13-15) to indicate its position underneath the flap 256. Additional dashed lines are used in FIG. 12 to show portions of components (e.g., sutures) that are disposed underneath the flap 256 (or extending along the first surface 266 of the flap 256 which is opposite the second surface 272 which is visible in FIG. 12).

The bottom primary suture tails 244a, 244b can be extended through the attachment member 204 (e.g., through the central portion 262) to opposite sides of the radially outward facing surface 214 which is disposed underneath the flap 256 in FIG. 12 (thus these suture tails are shown with dashed lines below the marker 250 in FIG. 12). The bottom primary suture tail 244a extends from a location 289 that is disposed axially offset from the inflow end 257 of the marker 250 and laterally offset from the first lateral side 251 of the marker 250 in a first direction (e.g., below and to the right of the marker 250 in the view of FIG. 12), toward the inflow end 257 of the marker 250. In some examples, the location 289 can be laterally offset from a central longitudinal axis of the marker 250 instead of the first lateral side 251 (for example, the location 289 may be laterally aligned or adjacent to the first lateral side 251 of the marker 250).

The bottom primary suture tail 244a then extends from underneath the marker 250 (or the first radial surface 261 of the marker 250, as shown in FIG. 9), through the first aperture 252 in the marker 250 and the flap 256, across the radially outward facing surface of the flap 256 (second surface 272) between the first aperture 252 and the second aperture 254, back through the flap 256 and the second aperture 254, underneath the marker 250, and back through the flap 256 to the radially outward facing surface of the flap 256. The bottom primary suture tail 244a then extends across the second surface 272 of the flap 256 to a location that is disposed axially offset from the outflow end 255 of the marker 250 and laterally offset from the first lateral side 251 of the marker 250 (or the central longitudinal axis of the marker 250) in the first direction (above and to the right of the marker 250 in the view of FIG. 12). This location is denoted by a first knot 288 which can be formed by tying the bottom primary suture tail 244a with the top secondary suture tail 236a on the radially outward facing surface of the flap 256. For example, the top secondary suture tail 236a can extend around or through the flap 256 to be tied with the bottom primary suture tail 244a. In this way, the bottom primary suture tail 244a forms an approximate forward “C” shape through the marker 250 and across the attachment member 204.

Similarly, the bottom primary suture tail 244b extends from a location 291 that is disposed axially offset from the inflow end 257 of the marker 250 and laterally offset from the second lateral side 253 (or central longitudinal axis) of the marker 250 in a second direction (e.g., below and to the left of the marker 250 in the view of FIG. 12), toward the inflow end 257 of the marker 250. The bottom primary suture tail 244b then extends from underneath the marker 250, through the first aperture 252 in the marker 250 and the flap 256, across the radially outward facing surface of the flap 256 between the first aperture 252 and the second aperture 254, back through the flap 256 and the second aperture 254, underneath the marker 250, and back through the flap 256 to the radially outward facing surface of the flap 256. The bottom primary suture tail 244b then extends across the second surface 272 of the flap 256 to a location that is disposed axially offset from the outflow end 255 of the marker 250 and laterally offset from the second lateral side 253 (or central longitudinal axis) of the marker 250 in the second direction (above and to the left of the marker 250 in the view of FIG. 12). This location is denoted by a second knot 290 which can be formed by tying the bottom primary suture tail 244b with the top secondary suture tail 236b on the radially outward facing surface of the flap 256. For example, the top secondary suture tail 236b can extend around or through the flap 256 to be tied with the bottom primary suture tail 244b. In this way, the bottom primary suture tail 244b forms an approximate reverse “C” shape through the marker 250 and across the attachment member 204.

In alternate examples, instead of forming knots 288 and 290, the bottom primary sutures tails 244a, 244b can be connected to the outflow portion of the attachment member 204 in a different way (other than be tied with the top secondary suture tails 236a, 236b), such as by gluing, mechanically fixing, or otherwise connecting ends of the bottom primary sutures 244a, 244b to the attachment member at the same points on the attachment member 204 (e.g., replacing the knots 288 and 290 by alternate connections to the attachment member 204).

The sewing configuration shown in FIG. 12 can be referred to as a non-crossing X pattern. However, in some examples, the portions of the bottom primary suture tails 244a, 244b that extend over the flap 256 between the first aperture 252 and the second aperture 254 can overlap and/or cross over one another while still maintaining the overall non-crossing X pattern (e.g., ends of the bottom primary suture tail 244a are both on the same, first lateral side of the attachment member 204 while ends of the bottom primary suture tail 244b are both on the same, second lateral side of the attachment member 204).

In this way, the bottom primary suture tails 244a, 244b are used to attach the marker 250 to the attachment member 204 and suspend the marker 250 across the cell of the frame (e.g., in lieu of the sutures 281a, 281b shown in FIGS. 10 and 11). In alternate examples, different sutures can be used to attach the marker 250 to the attachment member 204, in a similar manner to that described above with reference to FIG. 12. For example, in some instances, sutures from a location above or axially offset from the outflow end 255 of the marker 250 (such as the top secondary suture tails 236a, 236b or the top primary suture tails 234a, 234b) can be routed from above the marker 250, through the flap 256 and marker 250, to knots that are formed below the marker 250 (e.g., at locations 289 and 291). In this way, instead of routing sutures from the inflow end to the outflow end of the marker 250 (as shown in FIG. 12), the reverse can be possible where the sutures are routed from the outflow end to the inflow of the marker 250.

Returning to FIG. 12, in some examples, the top primary suture tails 234a, 234b can extend through the attachment member to the second surface 272 of the flap 256, on opposite sides of the central longitudinal axis 205 of the attachment member 204. The top primary suture tails 234a, 234b can then be used to form whip stitches 292 around struts 72 defining the cell 74 and through the perimeter of the main body portion 212 of the attachment member 204, thereby coupling the attachment member 204 to the frame 52 of the prosthetic valve (as shown in FIGS. 14A, 14B, and 15).

A third knot 294 can be formed on the second surface 272 of the flap 256, axially offset from the inflow end 257 of the marker 250 (for example, at an inflow end of the flap 256 which can be a free end of the flap 256). The third knot 294 can be formed from the bottom secondary suture tails 240a, 240b (FIG. 13). The third knot 994 can secure the flap 256 against the main body portion 212 of the attachment member 204.

Since the suture tails attaching the attachment member 204 to the struts 72 of the frame 52 (the top primary suture tails 234a, 234b) are different than the suture tails attaching the marker 250 to the attachment member 204 (the bottom primary suture tails 244a, 244b), the marker 250 is not directly attached to the struts 72 defining the cell 74. Further, by forming the knots 288 and 290 shown in FIGS. 12 and 13 at an axially offset location (e.g., above) from the marker 250, the marker 250 can be allowed to move as the frame elongates (during crimping) to the compressed state (FIG. 15) and shortens (during expansion) to the expanded state (FIGS. 14A and 14B). As a result, the marker 250 does not limit elongation of the frame 52 during radial compression of the prosthetic valve. This can result in a minimized crimp profile for the prosthetic valve, thereby reducing push forces felt by a user advancing the prosthetic valve crimped onto the delivery apparatus to an implantation site. Further, due to the non-crossing X pattern (or forward and reverse C pattern) of the sutures attaching the marker 250 to the attachment member 204, suture tension of the sutures holding the marker 250 against the attachment member 204, within the cell 74, remains relatively constant in both the radially expanded state (FIGS. 14A and 14B) and the radially compressed state (FIG. 15). As a result, the marker 250 remains within or close to the cell on the exterior of the frame, without bulging radially outward (e.g., as shown in FIG. 14B).

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein, Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve comprising: a frame including a plurality of struts forming a plurality of cells of the frame arranged between an inflow end and an outflow end of the frame, wherein the frame is radially collapsible to a collapsed configuration and radially expandable to an expanded configuration; a plurality of leaflets arranged within the frame; at least one commissure comprising an attachment member and commissure tabs of two adjacent leaflets coupled to the attachment member, wherein the attachment member is arranged across a specified cell of the plurality of cells and is attached to struts of the frame defining the specified cell; and a radiopaque marker suspended within the specified cell and disposed against the attachment member by sutures that extend from a first portion of the attachment member that is disposed adjacent to a first end of the marker, through one or more apertures in the marker, and to a second portion of the attachment member that is disposed adjacent to a second end of the marker, wherein the sutures form two knots in the second portion of the attachment member that are disposed on opposite sides of the marker such that the sutures form a non-crossing X-pattern through the marker and across the attachment member.

Example 2. The prosthetic heart valve of any example herein, particularly example 1, wherein the first end of the marker is directed toward the inflow end of the frame and the second end of the marker is directed toward the outflow end of the frame.

Example 3. The prosthetic heart valve of any example herein, particularly either example 1 or example 2, wherein the specified cell is disposed at the outflow end of the frame, wherein the second portion of the attachment member is disposed at an outflow end of the specified cell, and wherein the first portion of the attachment member is disposed at an inflow end of the specified cell.

Example 4. The prosthetic heart valve of any example herein, particularly example 1, wherein the first end of the marker is directed toward the outflow end of the frame and the second end of the marker is directed toward the inflow end of the frame.

Example 5. The prosthetic heart valve of any example herein, particularly either example 1 or example 4, wherein the specified cell is disposed at the outflow end of the frame, wherein the second portion of the attachment member is disposed at an inflow end of the specified cell, and wherein the first portion of the attachment member is disposed at an outflow end of the specified cell.

Example 6. The prosthetic heart valve of any example herein, particularly any one of examples 1-5, wherein a first distance between each knot of the two knots and the second end of the marker when the frame is in the expanded configuration and a second distance between each knot and the second end of the marker when the frame is in the collapsed configuration are the same.

Example 7. The prosthetic heart valve of any example herein, particularly any one of examples 1-6, wherein the sutures include a first suture that extends on a first radial side of the marker from the first portion of the attachment member to a first aperture in the marker, through the first aperture, from the first aperture to a second aperture in the marker on a second radial side of the marker, through the second aperture to the first radial side of the marker, and from the second aperture to the second portion of the attachment member where a first knot if formed with the first suture, and wherein the first knot is disposed axially offset from the second end of the marker and laterally offset in a first lateral direction from a first lateral side of the marker.

Example 8. The prosthetic heart valve of any example herein, particularly example 7, wherein the first radial side is a radially inward facing surface and the second radial side is a radially outward facing surface of the marker, relative to a central longitudinal axis of the prosthetic heart valve, and wherein the first aperture and the second aperture extend between the first radial side and the second radial side of the marker.

Example 9. The prosthetic heart valve of any example herein, particularly either example 7 or example 8, wherein the sutures include a second suture that extends on the first radial side of the marker from the first portion of the attachment member to the first aperture in the marker, through the first aperture, from the first aperture to the second aperture in the marker on the second radial side of the marker, through the second aperture to the first radial side of the marker, and from the second aperture to the second portion of the attachment member where a second knot if formed with the second suture, and wherein the second knot is disposed axially offset from the second end of the marker and laterally offset in second lateral direction from a second lateral side of the marker, the second lateral direction opposite the first lateral direction.

Example 10. The prosthetic heart valve of any example herein, particularly example 9, wherein the first suture extends from a first lateral side of the first portion of the attachment member and the second suture extends from a second lateral side of the first portion of the attachment member, the first and second lateral sides being opposite one another across a central longitudinal axis of the attachment member.

Example 11. The prosthetic heart valve of any example herein, particularly either example 9 or example 10, wherein the first knot is formed with the first suture and a third suture, and wherein the first suture and the third suture are suture tails from opposite ends of two different stitch lines that extend across a first side of the commissure to secure the commissure tabs and the attachment member together.

Example 12. The prosthetic heart valve of any example herein, particularly any one of examples 9-11, wherein the second knot is formed with the second suture and a fourth suture, and wherein the second suture and the fourth suture are suture tails from opposite ends of two different stitch lines that extend across a second side of the commissure to secure the commissure tabs and the attachment member together.

Example 13. The prosthetic heart valve of any example herein, particularly any one of examples 7-12, wherein the attachment member is attached to struts of the frame defining the specified cell with fifth and sixth sutures that extend through the attachment member and wrap around the struts and extend through a periphery of the attachment member around the specified cell.

Example 14. The prosthetic heart valve of any example herein, particularly any one of examples 1-13, where the attachment member comprises an elongate flap extending axially outward from a main body portion of the attachment member, wherein the marker is disposed against a first surface of the flap, and wherein the flap is folded over the main body portion such that the marker is disposed between the flap and the main body portion such that a second surface of the flap that is disposed opposite the first surface of the flap becomes a radially outward facing surface of the attachment member.

Example 15. The prosthetic heart valve of any example herein, particularly example 14, wherein the two knots are formed on the second surface of the flap, and wherein a third knot is formed with seventh and eighth sutures that extend through the flap and tie together on the second surface of the flap such that the flap is secured to the main body portion of the attachment member.

Example 16. The prosthetic heart valve of any example herein, particularly any one of examples 1-15, wherein the attachment member comprises one or more of a fabric and flexible polymeric material.

Example 17. The prosthetic heart valve of any example herein, particularly any one of examples 1-16, wherein the marker comprises tantalum.

Example 18. The prosthetic heart valve of any example herein, particularly any one of examples 1-17, wherein the marker has an oval shape with its longest dimension arranged in an axial direction of the prosthetic heart valve.

Example 19. The prosthetic heart valve of any example herein, particularly any one of examples 1-18, wherein the marker comprises two apertures spaced axially apart from one another.

Example 20. The prosthetic heart valve of any example herein, particularly any one of examples 1-19, wherein the prosthetic heart valve is balloon-expandable and configured to expand from the collapsed configuration to the expanded configuration in response to pressure applied to the frame by inflation of a balloon of a delivery apparatus.

Example 21. An assembly comprising: a delivery apparatus; and an implantable prosthetic heart valve that is radially collapsible to a collapsed configuration and radially expandable to an expanded configuration, the prosthetic heart valve comprising: a frame including a plurality of struts forming a plurality of cells of the frame arranged between an inflow end and an outflow end of the frame, wherein the frame is radially collapsible to a collapsed configuration and radially expandable to an expanded configuration; a plurality of leaflets arranged within the frame; at least one commissure comprising an attachment member and commissure tabs of two adjacent leaflets coupled to the attachment member, wherein the attachment member is arranged across a specified cell of the plurality of cells of the frame and is attached to struts of the frame defining the specified cell; and a radiopaque marker secured against the attachment member by first and second sutures that extend from opposite sides of a first portion of the attachment member that is disposed adjacent to a first end of the marker, through one or more apertures in the marker, and to a second portion of the attachment member that is disposed adjacent to a second end of the marker, wherein the first and second sutures form first and second knots, respectively, in the second portion of the attachment member that are disposed on opposite sides of the marker such that tension of the first and second sutures against the marker is maintained when the frame is in both the collapsed configuration and the expanded configuration; wherein the collapsed prosthetic heart valve can be mounted around a valve mounting portion of a distal end portion of the delivery apparatus and radially expanded to the expanded configuration with the delivery apparatus inside a patient's body.

Example 22. The assembly of any example herein, particularly example 21, wherein the first and second sutures form a non-crossing X-pattern through the marker and across the attachment member.

Example 23. The assembly of any example herein, particularly either example 21 or example 22, wherein the first suture forms a forward C shape and the second suture forms a reverse C shape across the attachment member.

Example 24. The assembly of any example herein, particularly any one of examples 21-23, wherein the first end of the marker is disposed closer to the inflow end of the frame than the second end of the marker, and wherein the second end of the marker is disposed closer to the outflow end of the frame than the first end of the marker.

Example 25. The assembly of any example herein, particularly any one of examples 21-24, wherein the specified cell is disposed at the outflow end of the frame, wherein the second portion of the attachment member is disposed at an outflow end of the specified cell, and wherein the first portion of the attachment member is disposed at an inflow end of the specified cell.

Example 26. The assembly of any example herein, particularly example 25, wherein the marker is suspended within an intermediate portion of the specified cell by the first and second sutures, the intermediate portion disposed between the inflow end and the outflow end of the specified cell.

Example 27. The assembly of any example herein, particularly any one of examples 21-23, wherein the first end of the marker is disposed closer to the outflow end of the frame than the second end of the marker, and wherein the second end of the marker is disposed closer to the inflow end of the frame than the first end of the marker.

Example 28. The assembly of any example herein, particularly any one of examples 21-27, wherein a first distance defined between each one of the first and second knots and the second end of the marker when the frame is in the expanded configuration is equal to a second distance defined between each one of the first and second knots and the second end of the marker when the frame is in the collapsed configuration.

Example 29. The assembly of any example herein, particularly any one of examples 21-28, wherein the first suture extends from a first location on the first portion of the attachment member to a first aperture in the marker, through the first aperture, across the marker between the first aperture and a second aperture in the marker, through the second aperture, and to the second portion of the attachment member where the first knot if formed with the first suture, wherein the first location is disposed axially offset from the first end of the marker and laterally offset in a first lateral direction from a first lateral side of the marker, and wherein the first knot is disposed axially offset from the second end of the marker and laterally offset in the first lateral direction from the first lateral side of the marker.

Example 30. The assembly of any example herein, particularly example 29, wherein the second suture extends from a second location on the first portion of the attachment member to the first aperture in the marker, through the first aperture, across the marker between the first aperture and the second aperture in the marker, through the second aperture, and to the second portion of the attachment member where the second knot if formed with the second suture, wherein the second location is disposed axially offset from the first end of the marker and laterally offset in a second lateral direction from a second lateral side of the marker, and wherein the second knot is disposed axially offset from the second end of the marker and laterally offset in the second lateral direction from the second lateral side of the marker, the second lateral direction opposite the first lateral direction.

Example 31. The assembly of any example herein, particularly example 30, wherein the first knot is formed with the first suture and a third suture, wherein the second knot is formed with the second suture and a fourth suture, and wherein the attachment member is attached to struts of the frame defining the specified cell with fifth and sixth sutures that extend through the attachment member and wrap around the struts and extend through a periphery of the attachment member around the specified cell.

Example 32. The assembly of any example herein, particularly example 31, wherein the first and second sutures are suture tails extending from a first end of two primary stitch lines disposed on opposite sides of the commissure that secure the commissure tabs and the attachment member together, wherein the third and fourth sutures are suture tails extending from a second end of two secondary stitch lines disposed on opposite sides of the commissure that secure the commissure tabs and the attachment member together, and wherein the fifth and sixth sutures are suture tails extending from a second end of the two primary stitch lines.

Example 33. The assembly of any example herein, particularly any one of examples 21-32, where the attachment member comprises a main body portion and an elongate flap extending axially outward from the main body portion, wherein the marker is disposed against a first surface of the flap, and wherein the flap is folded over the main body portion such that the marker is disposed between the flap and the main body portion of the flap such that a second surface of the flap that is disposed opposite the first surface of the flap becomes a radially outward facing surface of the attachment member on an exterior of the specified cell.

Example 34. The assembly of any example herein, particularly example 33, wherein the first and second knots are formed on the second surface of the flap, and wherein a third knot is formed with seventh and eighth sutures that extend through the flap and tie together on the second surface of the flap such that the flap is secured to the main body portion of the attachment member.

Example 35. The assembly of any example herein, particularly any one of examples 21-34, wherein the attachment member comprises one or more of a fabric and flexible polymeric material.

Example 36. The assembly of any example herein, particularly any one of examples 21-35, wherein the marker comprises tantalum.

Example 37. The assembly of any example herein, particularly any one of examples 21-36, wherein the marker has an oval shape with its longest dimension arranged in an axial direction of the prosthetic heart valve.

Example 38. The assembly of any example herein, particularly any one of examples 21-37, wherein the marker comprises two apertures spaced axially apart from one another.

Example 39. The assembly of any example herein, particularly any one of examples 21-38, wherein the delivery apparatus comprises a balloon, and wherein the collapsed prosthetic heart valve can be mounted around the balloon and radially expanded to the expanded configuration with the balloon inside the patient's body.

Example 40. The assembly of any example herein, particularly example 39, wherein the prosthetic heart valve is radially crimped around the balloon into the collapsed configuration such that the collapsed prosthetic heart valve is disposed radially outward of the balloon.

Example 41. The assembly of any example herein, particularly example 39, wherein the prosthetic heart valve is radially crimped around the delivery apparatus into the collapsed configuration such that it is axially offset from the balloon.

Example 42. The assembly of any example herein, particularly any one of examples 21-38, wherein the prosthetic heart valve is self-expandable and contained around the valve mounting portion of the delivery apparatus in the collapsed configuration by a moveable delivery capsule of the delivery apparatus.

Example 43. A method comprising: extending sutures from opposite sides of a first portion of a commissure attachment member, through one or more apertures in a marker disposed on the attachment member, and to opposite sides of a second portion of the attachment member, wherein the first portion is disposed adjacent to a first end of the marker and the second portion is disposed adjacent to a second end of the marker, the second end arranged opposite the first end in an axial direction, and wherein the attachment member forms a commissure with two adjacent leaflets of a prosthetic heart valve and is arranged across a cell of a frame of the prosthetic heart valve; forming knots with the sutures on opposite sides of the second portion of the attachment member such that the marker is suspended within the cell; and radially compressing the prosthetic heart valve onto a delivery apparatus from a radially expanded configuration to a radially compressed configuration, and moving the knots axially away from the marker and laterally toward one another in response to axially elongating the frame during radial compression of the prosthetic heart valve such that a distance along the sutures between the knots and the marker is maintained between the radially expanded and radially compressed configurations.

Example 44. The method of any example herein, particularly example 43, wherein the sutures form a non-crossing X-pattern from the first portion to the second portion of the attachment member.

Example 45. The method of any example herein, particularly either example 43 or claim 44, wherein the knots include a first knot that is laterally offset from the second end of the marker in a first direction and second knot that is laterally offset from the second end of the marker in a second direction that is opposite the first direction.

Example 46. The method of any example herein, particularly any one of examples 43-45, wherein extending the sutures and forming the knots includes: extending a first suture from a first location on the first portion of the attachment member, to a first aperture in the marker, through the first aperture, across the marker between the first aperture and a second aperture in the marker, through the second aperture, and to a second location on the second portion of the attachment member, and forming a first knot at the second location, wherein the first location is disposed axially offset from the first end of the marker and laterally offset in a first lateral direction from the first end of the marker, and wherein the second location is disposed axially offset from the second end of the marker and laterally offset in the first lateral direction from the second end of the marker; and extending a second suture from a third location on the first portion of the attachment member, to the first aperture in the marker, through the first aperture, across the marker between the first aperture and the second aperture in the marker, through the second aperture, and to a fourth location on the second portion of the attachment member, and forming a second knot at the fourth location, wherein the third location is disposed axially offset from the first end of the marker and laterally offset in a second lateral direction from the first end of the marker, and wherein the fourth location is disposed axially offset from the second end of the marker and laterally offset in the second lateral direction from the second end of the marker.

Example 47. The method of any example herein, particularly example 46, wherein the first and second locations are axially offset from and laterally aligned with one another, and wherein the third and fourth locations are axially offset from and laterally aligned with one another.

Example 48. The method of any example herein, particularly any one of examples 43-47, wherein the commissure is formed by attaching commissure tabs of the two adjacent leaflets to a radially inward facing surface of the attachment member, and wherein the knots are formed on a radially outward facing surface of the attachment member.

Example 49. The method of any example herein, particularly example 48, further comprising forming the commissure by extending a plurality of sutures through the commissure tabs and the attachment member to form a plurality of stitch lines that secure the attachment member and commissure tabs together and result in suture tails extending from both ends of each stitch line of the plurality of stitch lines, and wherein extending the sutures from opposite sides of the first portion of the attachment member includes extending two first suture tails from separate stitch lines of the plurality of stitch lines through the attachment member to the radially outward facing surface of the attachment member and then extending the two first suture tails through the one or more apertures in the marker and to opposite sides of the second portion of the attachment member.

Example 50. The method of any example herein, particularly example 49, wherein forming the knots includes forming the knots with the two first suture tails and two second suture tails from additional separate stitch lines of the plurality of stitch lines.

Example 51. The method of any example herein, particularly any one of examples 43-50, wherein the first portion of the attachment member is disposed at an inflow end of the cell and the second portion of the attachment member is disposed at an outflow end of the cell, and wherein the marker is disposed on an intermediate portion of the attachment member that is disposed between the first portion and the second portion.

Example 52. The method of any example herein, particularly any one of examples 43-50, wherein the first portion of the attachment member is disposed at an outflow end of the cell and the second portion of the attachment member is disposed at an inflow end of the cell, and wherein the marker is disposed on an intermediate portion of the attachment that is disposed between the first portion and the second portion.

Example 53. The method of any example herein, particularly any one of examples 43-52, further comprising attaching the attachment member to struts of the frame that define the cell by extending additional sutures through the attachment member around the struts, around a perimeter of the attachment member, and wherein the marker is not directly attached to the struts.

Example 54. The method of any example herein, particularly any one of examples 43-53, wherein the marker moves freely as the prosthetic heart valve is radially compressed and the frame axially elongates but remains taught against the attachment member.

Example 55. The method of any example herein, particularly any one of examples 43-54, wherein the marker is a radiopaque marker comprising a radiopaque material.

Example 56. The method of any example herein, particularly any one of examples 43-55, wherein the marker comprises tantalum.

Example 57. The method of any example herein, particularly any one of examples 43-56, wherein the marker comprises two or more apertures.

Example 58. A method comprising: securing commissure tabs of two adjacent leaflets of a prosthetic heart valve to a first surface of an attachment member to form a commissure, wherein the attachment member is configured to be arranged across a cell of a frame of the prosthetic heart valve; positioning a radiopaque marker against a second surface of the attachment member that is disposed opposite the first surface; extending a first suture from a first location on the attachment member, through at least two apertures in the marker, and to a second location on the attachment member and forming a first knot with the first suture that secures the first suture to the first location of the attachment member, wherein the first location is offset in a first axial direction and a first lateral direction from a first end of the marker and the second location is offset in a second axial direction and the first lateral direction from a second end of the marker; extending a second suture from a third location on the attachment member, through the at least two apertures in the marker, and to a fourth location on the attachment member and forming a second knot with the second suture that secures the second suture to the second location of the attachment member, wherein the third location is offset in a first axial direction and a second lateral direction from the first end of the marker and the fourth location is offset in the second axial direction and the second lateral direction from the second end of the marker; and securing the attachment member to struts of the frame that define the cell with a third suture such that the marker is disposed within the cell and not directly attached to the struts defining the cell.

Example 59. The method of any example herein, particularly example 58, further comprising radially compressing the prosthetic heart valve onto a delivery apparatus from a radially expanded configuration to a radially compressed configuration, and moving the first and second knots axially away from the marker and laterally toward one another in response to axially elongating the frame during radial compression of the prosthetic heart valve such that a tension in the first suture and the second suture is maintained between the radially expanded configuration and the radially compressed configuration.

Example 60. The method of any example herein, particularly either example 58 or example 59, wherein the first end of the marker is directed toward an inflow end of the frame and the second end of the marker is directed toward an outflow end of the frame.

Example 61. The method of any example herein, particularly either example 58 or example 59, wherein the first end of the marker is directed toward an outflow end of the frame and the second end of the marker is directed toward an inflow end of the frame.

Example 62. The method of any example herein, particularly any one of examples 58-61, wherein the first and second locations are laterally aligned with one another, and wherein the third and fourth locations are laterally aligned with one another.

Example 63. The method of any example herein, particularly any one of examples 58-62, wherein securing commissure tabs of two adjacent leaflets to the first surface of the attachment member to form the commissure includes forming a plurality of stitch lines through the commissure tabs and the attachment member, axially across the commissure, with a plurality of sutures, wherein the first and second sutures are first and second suture tails extending from a first end of first and second stitch lines, respectively, of the plurality of stitch lines, and further comprising extending the first and second sutures through the attachment member, from the first surface to the second surface, at the first and third locations on the attachment member.

Example 64. The method of any example herein, particularly example 63, wherein forming the first knot includes forming the first knot with the first suture and a third suture tail that extends through the attachment member, the third suture tail extending from a third stitch line of the plurality of stitch lines, and wherein forming the second knot includes forming the second knot with the second suture and a fourth suture tail that extends through the attachment member, the fourth suture tail extending from a fourth stitch line of the plurality of stitch lines.

Example 65. The method of any example herein, particularly either example 63 or example 64, wherein the third suture is a fifth suture tail extending from a second end of the first stitch line, and wherein securing the attachment member to struts of the frame that define the cell includes securing the attachment member to struts of the frame that define the cell with the fifth suture tail and a sixth suture tail that extends through the attachment member from the second end of the second stitch line.

Example 66. The method of any example herein, particularly any one of examples 58-65, wherein the marker comprises tantalum.

Example 67. The method of any example herein, particularly any one of examples 58-66, wherein the marker comprises two apertures, and wherein the first and second sutures extend over an outer surface of the marker between the two apertures and extend over an inner surface of the marker between each respective aperture of the two apertures and the first and second ends of the marker.

Example 68. The method of any example herein, particularly any one of examples 58-67, wherein positioning the marker against a second surface of the attachment member that is disposed opposite the first surface includes positioning the marker between the second surface of a main body portion of the attachment member and an elongate flap that extends from the main body portion and is folded over the second surface such that it covers the marker, and wherein the first and second sutures extend through the flap.

Example 69. The method of any example herein, particularly example 68, further comprising forming a third knot on an outer surface of the flap with fourth and fifth sutures such that the flap is held against the second surface of the main body portion of the attachment member.

Example 70. A prosthetic heart valve, comprising: a frame including a plurality of struts forming a plurality of cells of the frame arranged between an inflow end and an outflow end of the frame, wherein the frame is radially collapsible to a collapsed configuration and radially expandable to an expanded configuration; a plurality of leaflets arranged within the frame; at least one commissure comprising an attachment member and commissure tabs of two adjacent leaflets coupled to the attachment member, wherein the attachment member is arranged across a specified cell of the plurality of cells of the frame and attached to struts of the frame forming the specified cell; and a radiopaque marker suspended within the specified cell with two sutures that extend through one or more apertures in the marker, the two sutures comprising: a first suture that extends from a first location on the attachment member that is disposed axially offset from a first end of the marker and laterally offset from the marker in a first direction, through the one or more apertures in the marker, and to a second location on the attachment member that is disposed axially offset from a second end of the marker and laterally offset from the marker in the first direction; and a second suture that extends from a third location on the attachment member that is disposed axially offset from the first end of the marker and laterally offset from the marker in a second direction, through the one or more apertures in the marker, and to a fourth location on the attachment member that is disposed axially offset from the second end of the marker and laterally offset from the marker in the second direction.

Example 71. The prosthetic heart valve of any example herein, particularly example 70, wherein the first and third locations are in an inflow end portion of the attachment member that is connected to an inflow end of the specified cell, wherein the second and fourth locations are in an outflow end portion of the attachment member that is connected to an outflow end of the specified cell, and wherein the marker is disposed against an intermediate portion of the attachment member, the intermediate portion defined between and separating the inflow end portion and the outflow end portion of the attachment member.

Example 72. The prosthetic heart valve of any example herein, particularly example 70, wherein the first and third locations are in an outflow end portion of the attachment member that is connected to an outflow end of the specified cell, wherein the second and fourth locations are in an inflow end portion of the attachment member that is connected to an inflow end of the specified cell, and wherein the marker is disposed against an intermediate portion of the attachment member, the intermediate portion defined between and separating the inflow end portion and the outflow end portion of the attachment member.

Example 73. The prosthetic heart valve of any example herein, particularly any one of examples 70-72, wherein the first and second locations are spaced axially apart from one another and laterally aligned with one another.

Example 74. The prosthetic heart valve of any example herein, particularly any one of examples 70-73, wherein the third and fourth locations are spaced axially apart from one another and laterally aligned with one another.

Example 75. The prosthetic heart valve of any example herein, particularly any one of examples 70-74, wherein the one or more apertures in the marker includes a first aperture and second aperture that are spaced axially apart from one another.

Example 76. The prosthetic heart valve of any example herein, particularly any one of examples 70-75, wherein a first knot is formed at the second location with the first suture and a third suture, and a second knot is formed at the fourth location with the second suture and a fourth suture such that the first and second sutures retain the marker against the attachment member with a specified suture tension.

Example 77. The prosthetic heart valve of any example herein, particularly example 76, wherein the specified suture tension is maintained when the prosthetic heart valve is the collapsed configuration and the expanded configuration.

Example 78. The prosthetic heart valve of any example herein, particularly either example 76 or example 77, wherein a first length of the first and second sutures between the second end of the marker and the corresponding first and second knots when the frame is in the expanded configuration is equal to a second length of the first and second sutures between the second end of the marker and the corresponding first and second knots when the frame is in the collapsed configuration.

Example 79. The prosthetic heart valve of any example herein, particularly any one of examples 76-78, wherein the first suture and the third suture are suture tails from two different stitch lines that extend across a first side of the commissure to secure the commissure tabs and the attachment member together, and wherein the second suture and the fourth suture are suture tails from two different stitch lines that extend across a second side of the commissure to secure the commissure tabs and the attachment member together.

Example 80. The prosthetic heart valve of any example herein, particularly any one of examples 76-79, wherein the attachment member is attached to struts of the frame defining the specified cell with fifth and sixth sutures that extend through the attachment member and wrap around the struts and extend through a periphery of the attachment member around the specified cell.

Example 81. The prosthetic heart valve of any example herein, particularly any one of examples 76-80, where the attachment member comprises an elongate flap extending axially outward from a main body portion of the attachment member, wherein the marker is disposed against a first surface of the flap, and wherein the flap is folded over the main body portion such that the marker is disposed between the flap and the main body portion of the flap such that a second surface of the flap that is disposed opposite the first surface of the flap becomes a radially outward facing surface of the attachment member.

Example 82. The prosthetic heart valve of any example herein, particularly example 81, wherein the first and second knots are formed on the second surface of the flap, and wherein a third knot is formed with seventh and eighth sutures that extend through the flap and tie together on the second surface of the flap such that the flap is secured to the main body portion of the attachment member.

Example 83. The prosthetic heart valve of any example herein, particularly any one of examples 70-82, wherein the attachment member comprises one or more of a fabric and flexible polymeric material.

Example 84. The prosthetic heart valve of any example herein, particularly any one of examples 70-83, wherein the marker comprises tantalum.

Example 85. The prosthetic heart valve of any example herein, particularly any one of examples 70-84, wherein the prosthetic heart valve is balloon-expandable and configured to expand from the collapsed configuration to the expanded configuration in response to pressure applied to the frame by inflation of a balloon of a delivery apparatus.

Example 86. A commissure attachment member comprising: a main body portion; and a radiopaque marker secured to the main body portion by sutures that extend from a first portion of the main body portion of the attachment member that is disposed adjacent to a first end of the marker, through one or more apertures in the marker, and to a second portion of the main body portion of the attachment member that is disposed adjacent to a second end of the marker, wherein the sutures form two connections to the second portion of the attachment member that are disposed on opposite sides of the marker such that the sutures form a non-crossing X-pattern through the marker and across the main body portion of the attachment member.

Example 87. The commissure attachment member of any example herein, particularly example 86, wherein the marker is disposed against a third portion of the main body portion that is disposed between the first portion and the second portion of the main body portion.

Example 88. The commissure attachment member of any example herein, particularly either example 86 or example 87, wherein the main body portion is movable between a first state and a second state, wherein the main body portion is elongated axially and compressed laterally in the second state relative to the first state, and wherein a first distance between each connection of the two connections and the second end of the marker when the main body portion is in the first state and a second distance between each connection and the second end of the marker when the main body portion is in the second state are the same.

Example 89. The commissure attachment member of any example herein, particularly any one of examples 86-88, wherein the two connections are two separate knots in the second portion of the attachment member that are formed with the sutures.

Example 90. The commissure attachment member of any example herein, particularly example 89, wherein the sutures include a first suture that extends on a first radial side of the marker from the first portion of the attachment member to a first aperture in the marker, through the first aperture, from the first aperture to a second aperture in the marker on a second radial side of the marker, through the second aperture to the first radial side of the marker, and from the second aperture to the second portion of the attachment member where a first knot if formed with the first suture, and wherein the first knot is disposed axially offset from the second end of the marker and laterally offset in a first lateral direction from a central longitudinal axis of the marker.

Example 91. The commissure attachment member of any example herein, particularly example 90, wherein the first aperture and the second aperture extend between the first radial side and the second radial side of the marker.

Example 92. The commissure attachment member of any example herein, particularly either example 90 or example 91, wherein the sutures include a second suture that extends on the first radial side of the marker from the first portion of the attachment member to the first aperture in the marker, through the first aperture, from the first aperture to the second aperture in the marker on the second radial side of the marker, through the second aperture to the first radial side of the marker, and from the second aperture to the second portion of the attachment member where a second knot if formed with the second suture, and wherein the second knot is disposed axially offset from the second end of the marker and laterally offset in second lateral direction from the central longitudinal axis of the marker, the second lateral direction opposite the first lateral direction.

Example 93. The commissure attachment member of any example herein, particularly example 92, wherein the first suture extends from a first lateral side of the first portion of the main body portion of the attachment member and the second suture extends from a second lateral side of the first portion of the main body portion of the attachment member, the first and second lateral sides being opposite one another across a central longitudinal axis of the attachment member.

Example 94. The commissure attachment member of any example herein, particularly either example 92 or example 93, wherein the first knot is formed with the first suture and a third suture that extends through the main body portion, from a first surface to a second surface of the main body portion, and wherein the first knot is formed on the second surface.

Example 95. The commissure attachment member of any example herein, particularly any one of examples 92-94, wherein the second knot is formed with the second suture and a fourth suture that extends through the main body portion, from a first surface to a second surface of the main body portion, and wherein the second knot is formed on the second surface.

Example 96. The commissure attachment member of any example herein, particularly any one of examples 86-95, wherein the main body portion of the attachment member is configured to be arranged across a cell of a prosthetic heart valve and secured to frame struts of the prosthetic heart valve that define the cell.

Example 97. The commissure attachment member of any example herein, particularly any one of examples 86-96, where the attachment member comprises an elongate flap extending axially outward from the main body portion of the attachment member, wherein the marker is disposed against a first surface of the flap, and wherein the flap is folded over the main body portion such that the marker is disposed between the flap and the main body portion such that a second surface of the flap that is disposed opposite the first surface of the flap becomes a radially outward facing surface of the attachment member.

Example 98. The commissure attachment member of any example herein, particularly example 97, wherein the two connections are two knots that are formed on the second surface of the flap, and wherein a third knot is formed with seventh and eighth sutures that extend through the flap and tie together on the second surface of the flap such that the flap is secured to the main body portion.

Example 99. The commissure attachment member of any example herein, particularly any one of examples 86-98, wherein the main body portion of the attachment member comprises one or more of a fabric and flexible polymeric material.

Example 100. The commissure attachment member of any example herein, particularly any one of examples 86-99, wherein the marker comprises tantalum.

Example 101. The commissure attachment member of any example herein, particularly any one of examples 86-100, wherein the marker has an oval shape with its longest dimension arranged in an axial direction of the prosthetic heart valve.

Example 102. The commissure attachment member of any example herein, particularly any one of examples 86-101, wherein the marker comprises two apertures spaced axially apart from one another.

Example 103. A commissure attachment member comprising: a main body portion; and a radiopaque marker secured to the main body portion by two sutures that extend through one or more apertures in the marker, the two sutures comprising: a first suture that extends from a first location on the main body portion of the attachment member that is disposed axially offset from a first end of the marker and laterally offset from the marker in a first direction, through the one or more apertures in the marker, and to a second location on the main body portion of the attachment member that is disposed axially offset from a second end of the marker and laterally offset from the marker in the first direction; and a second suture that extends from a third location on the main body portion of the attachment member that is disposed axially offset from the first end of the marker and laterally offset from the marker in a second direction, through the one or more apertures in the marker, and to a fourth location on the main body portion of the attachment member that is disposed axially offset from the second end of the marker and laterally offset from the marker in the second direction.

Example 104. A method comprising sterilizing the prosthetic heart valve, assembly, or commissure attachment member of any one of examples 1-103.

Example 105. A prosthetic heart valve of any one of examples 1-103, wherein the prosthetic heart valve is sterilized.

The features described herein with regard to any example can be combined with other features described in any one or more of the other examples, unless otherwise stated. For example, any one or more of the features of one prosthetic valve can be combined with any one or more features of another prosthetic valve.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A prosthetic heart valve comprising:
a frame including a plurality of struts forming a plurality of cells of the frame arranged between an inflow end and an outflow end of the frame, wherein the frame is radially collapsible to a collapsed configuration and radially expandable to an expanded configuration;
a plurality of leaflets arranged within the frame;
at least one commissure comprising an attachment member and commissure tabs of two adjacent leaflets coupled to the attachment member, wherein the attachment member is arranged across a specified cell of the plurality of cells and is attached to struts of the frame defining the specified cell; and
a radiopaque marker suspended within the specified cell and disposed against the attachment member by sutures that extend from a first portion of the attachment member that is disposed adjacent to a first end of the marker, through one or more apertures in the marker, and to a second portion of the attachment member that is disposed adjacent to a second end of the marker, wherein the sutures form two knots in the second portion of the attachment member that are disposed on opposite sides of the marker such that the sutures form a non-crossing X-pattern through the marker and across the attachment member.

2. The prosthetic heart valve of claim 1, wherein the first end of the marker is directed toward the inflow end of the frame and the second end of the marker is directed toward the outflow end of the frame.

3. The prosthetic heart valve of claim 1, wherein the specified cell is disposed at the outflow end of the frame, wherein the second portion of the attachment member is disposed at an outflow end of the specified cell, and wherein the first portion of the attachment member is disposed at an inflow end of the specified cell.

4. The prosthetic heart valve of claim 1, wherein a first distance between each knot of the two knots and the second end of the marker when the frame is in the expanded configuration and a second distance between each knot and the second end of the marker when the frame is in the collapsed configuration are the same.

5. The prosthetic heart valve of claim 1, where the attachment member comprises an elongate flap extending axially outward from a main body portion of the attachment member, wherein the marker is disposed against a first surface of the flap, and wherein the flap is folded over the main body portion such that the marker is disposed between the flap and the main body portion such that a second surface of the flap that is disposed opposite the first surface of the flap becomes a radially outward facing surface of the attachment member.

6. The prosthetic heart valve of claim 5, wherein the two knots are formed on the second surface of the flap, and wherein a third knot is formed with seventh and eighth sutures that extend through the flap and tie together on the second surface of the flap such that the flap is secured to the main body portion of the attachment member.

7. The prosthetic heart valve of claim 1, wherein the attachment member comprises one or more of a fabric and flexible polymeric material.

8. A method comprising:
extending sutures from opposite sides of a first portion of a commissure attachment member, through one or more apertures in a marker disposed on the attachment member, and to opposite sides of a second portion of the attachment member, wherein the first portion is disposed adjacent to a first end of the marker and the second portion is disposed adjacent to a second end of the marker, the second end arranged opposite the first end in an axial direction, and wherein the attachment member forms a commissure with two adjacent leaflets of a prosthetic heart valve and is arranged across a cell of a frame of the prosthetic heart valve;
forming knots with the sutures on opposite sides of the second portion of the attachment member such that the marker is suspended within the cell; and
radially compressing the prosthetic heart valve onto a delivery apparatus from a radially expanded configuration to a radially compressed configuration, and moving the knots axially away from the marker and laterally toward one another in response to axially elongating the frame during radial compression of the prosthetic heart valve such that a distance along the sutures between the knots and the marker is maintained between the radially expanded and radially compressed configurations.

9. The method of claim 8, wherein the knots include a first knot that is laterally offset from the second end of the marker in a first direction and second knot that is laterally offset from the second end of the marker in a second direction that is opposite the first direction.

10. The method of claim 8, wherein extending the sutures and forming the knots includes:
extending a first suture from a first location on the first portion of the attachment member, to a first aperture in the marker, through the first aperture, across the marker between the first aperture and a second aperture in the marker, through the second aperture, and to a second location on the second portion of the attachment member, and forming a first knot at the second location, wherein the first location is disposed axially offset from the first end of the marker and laterally offset in a first lateral direction from the first end of the marker, and wherein the second location is disposed axially offset from the second end of the marker and laterally offset in the first lateral direction from the second end of the marker; and
extending a second suture from a third location on the first portion of the attachment member, to the first aperture in the marker, through the first aperture, across the marker between the first aperture and the second aperture in the marker, through the second aperture, and to a fourth location on the second portion of the attachment member, and forming a second knot at the fourth location, wherein the third location is disposed axially offset from the first end of the marker and laterally offset in a second lateral direction from the first end of the marker, and wherein the fourth location is disposed axially offset from the second end of the marker and laterally offset in the second lateral direction from the second end of the marker.

11. The method of claim 10, wherein the first and second locations are axially offset from and laterally aligned with one another, and wherein the third and fourth locations are axially offset from and laterally aligned with one another.

12. The method of claim 8, wherein the commissure is formed by attaching commissure tabs of the two adjacent leaflets to a radially inward facing surface of the attachment member, and wherein the knots are formed on a radially outward facing surface of the attachment member.

13. The method of claim 12, further comprising forming the commissure by extending a plurality of sutures through the commissure tabs and the attachment member to form a plurality of stitch lines that secure the attachment member and commissure tabs together and result in suture tails extending from both ends of each stitch line of the plurality of stitch lines, and wherein extending the sutures from opposite sides of the first portion of the attachment member includes extending two first suture tails from separate stitch lines of the plurality of stitch lines through the attachment member to the radially outward facing surface of the attachment member and then extending the two first suture tails through the one or more apertures in the marker and to opposite sides of the second portion of the attachment member.

14. The method of claim 13, wherein forming the knots includes forming the knots with the two first suture tails and two second suture tails from additional separate stitch lines of the plurality of stitch lines.

15. The method of claim 8, wherein the marker moves freely as the prosthetic heart valve is radially compressed and the frame axially elongates but remains taught against the attachment member.

16. A commissure attachment member comprising:
a main body portion; and
a radiopaque marker secured to the main body portion by sutures that extend from a first portion of the main body portion of the attachment member that is disposed adjacent to a first end of the marker, through one or more apertures in the marker, and to a second portion of the main body portion of the attachment member that is disposed adjacent to a second end of the marker, wherein the sutures form two connections to the second portion of the attachment member that are disposed on opposite sides of the marker such that the sutures form a non-crossing X-pattern through the marker and across the main body portion of the attachment member.

17. The commissure attachment member of claim 16, wherein the main body portion is movable between a first state and a second state, wherein the main body portion is elongated axially and compressed laterally in the second state relative to the first state, and wherein a first distance between each connection of the two connections and the second end of the marker when the main body portion is in the first state and a second distance between each connection and the second end of the marker when the main body portion is in the second state are the same.

18. The commissure attachment member of claim 16, wherein the two connections are two separate knots in the second portion of the attachment member that are formed with the sutures.

19. The commissure attachment member of claim 18, wherein the sutures include:
a first suture that extends on a first radial side of the marker from the first portion of the attachment member to a first aperture in the marker, through the first aperture, from the first aperture to a second aperture in the marker on a second radial side of the marker, through the second aperture to the first radial side of the marker, and from the second aperture to the second portion of the attachment member where a first knot if formed with the first suture, and wherein the first knot is disposed axially offset from the second end of the marker and laterally offset in a first lateral direction from a central longitudinal axis of the marker; and
a second suture that extends on the first radial side of the marker from the first portion of the attachment member to the first aperture in the marker, through the first aperture, from the first aperture to the second aperture in the marker on the second radial side of the marker, through the second aperture to the first radial side of the marker, and from the second aperture to the second portion of the attachment member where a second knot if formed with the second suture, and wherein the second knot is disposed axially offset from the second end of the marker and laterally offset in second lateral direction from the central longitudinal axis of the marker, the second lateral direction opposite the first lateral direction.

20. The commissure attachment member of claim 16, wherein the main body portion of the attachment member is configured to be arranged across a cell of a prosthetic heart valve and secured to frame struts of the prosthetic heart valve that define the cell.
